(19) United States
(12) Patent Application Publication
(10) Pub. No.: US 2014/0328861 A1
(43) Pub. Date: Nov. 6, 2014

(54) Combination of CRTH2 Antagonist and a Proton Pump Inhibitor for the Treatment of Eosinophilic Esophagitis

(71) Applicant: Atopix Therapeutics Limited, London (GB)

(72) Inventors: Mark Anthony Payton, London (GB); Eric Roy Pettipher, London (GB)

(21) Appl. No.: 14/365,306

(22) PCT Filed: Dec. 14, 2012
(86) PCT No.: PCT/GB2012/000904

§ 371 Date: Jun. 13, 2014

Related U.S. Application Data
(60) Provisional application No. 61/576,640, filed on Dec. 16, 2011.

(51) Int. Cl. A61K 31/475; A61K 31/58; A61K 39/395; A61K 31/47; A61K 31/4439; C07K 16/24

(52) U.S. Cl. CPC A61K 31/475; A61K 31/4439; C07K 16/244; A61K 39/3955; A61K 31/47; A61K 31/58
USPC 424/158.1; 514/314; 514/338; 514/311; 514/171

(57) ABSTRACT

Disclosed are methods and compositions for preventing, treating, or ameliorating eosinophilic esophagitis (EoE) in an individual, comprising administering to the individual a therapeutically effective amount of at least one CRTH2 antagonist or a pharmaceutically acceptable salt thereof and at least one proton pump inhibitor (PPI) or a pharmaceutically acceptable salt thereof. Also disclosed are compositions comprising at least one CRTH2 antagonist or a pharmaceutically acceptable salt thereof and at least one proton pump inhibitor or a pharmaceutically acceptable salt thereof.

### BACKGROUND OF THE INVENTION

1. Field of the Invention

The present invention provides a method for the treatment of eosinophilic esophagitis by administering compositions comprising one or more CRTH2 antagonist compounds and one or more proton pump inhibitors.

2. Related Art

Eosinophilic esophagitis (EoE) is characterised by signs and symptoms related to esophageal dysfunction (Liacouras et al., J. Allergy Clin. Immunol. 128:3-20 (2011)). In adults these include dysphagia, chest pain, food impaction, and upper abdominal pain (Croese et al., Gastrointest. Endosc. 58:516-522 (2003); Furuta and Straumann, Aliment. Pharmacol. Ther. 24:173-182 (2006)). Clinical manifestations in children vary by age. Infants often present with feeding difficulties and failure to thrive, whereas school-aged children are more likely to present with vomiting or pain (Liacouras et al., 2011). Eosinophils are present histologically in biopsied esophageal tissue. EoE is considered to have an allergic etiology with 70% of EoE patients having current or past allergic disease or positive skin prick tests to food or other allergens (Blanchard and Rothenberg, Gastrointest. Endosc. Clin. N. Am. 18:133-43 (2008)). The signs and symptoms of EoE are generally resistant to proton pump inhibitor (PPI) therapy, although some patients do demonstrate a clinicopathological response to PPIs (Molina-Infante et al., Clin. Gastroenterol. Hepatol. 9:110-117 (2011)) and this has been described as “PPI-responsive esophageal eosinophilia” which may be differentiated from eosinophilic esophagitis based on response to PPIs (Liacouras et al., 2011). Topical corticosteroids, used ‘off-label’ in EoE, are very effective at reducing the eosinophilic load of the esophagus, a process thought to be mediated by the promotion of eosinophil apoptosis. Double-blind placebo-controlled trials have demonstrated that both fluticasone and budesonide are effective as induction treatments for reducing eosinophilic load and symptoms in both children and adults with EoE (Schaefer et al., Clin. Gastroenterol. Hepatol. 6:165-173 (2008); Konikoff et al., Gastroenterology 131:1381-1391 (2006); Dohil et al., Gastroenterology 139:418-429 (2010); Straumann et al., Gastroenterology 139:1526-1537 (2010)).

Although PPIs are not of general benefit in patients with EoE, many patients remain on these drugs to control acid reflux which may be secondary to inflammatory damage of the distal (lower) esophagus.

### BRIEF SUMMARY OF THE INVENTION

One aspect of the invention is to provide a method of preventing, treating, or ameliorating eosinophilic esophagitis (EoE) in an individual, comprising administering to the individual a therapeutically effective amount of at least one CRTH2 antagonist or a pharmaceutically acceptable salt thereof and at least one proton pump inhibitor (PPI) or a pharmaceutically acceptable salt thereof.

Another aspect of the invention is a composition comprising at least one CRTH2 antagonist or a pharmaceutically acceptable salt thereof and at least one proton pump inhibitor or a pharmaceutically acceptable salt thereof.

In one embodiment, the CRTH2 antagonist is a compound of general formula (I):

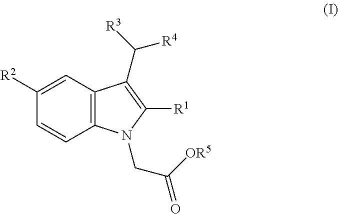

wherein

R¹ is C₁-C₆ alkyl;

R² is halogen;

R³ is aryl or heteroaryl optionally substituted with one or more substituents selected from halo, OH, CN, R⁶, COR⁶, CH₂R⁶, OR⁶, SR⁶, SO₂R⁶, or SO₂YR⁶;
R⁶ is C₁-C₆ alkyl, C₃-C₈ cycloalkyl, heterocyclyl, aryl, or heteroaryl, any of which may optionally be substituted with one or more substituents selected from halo, OH, CN, NO₂, C₁-C₆ alkyl, or O(C₁-C₆ alkyl); and
Y is NH or a straight or branched C₁-C₄ alkylene chain;

R⁴ is H or C₁-C₄ alkyl; and

R⁵ is hydrogen, C₁-C₆ alkyl, aryl, (CH₂)ₘOC(═O)C₁-C₆alkyl, ((CH₂)ₘO)ₙCH₂CH₂CH₂X, (CH₂)ₘN(R⁷)₂, or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;

or a pharmaceutically acceptable salt, hydrate, solvate, or complex thereof.

In one embodiment, R⁵ is hydrogen.

In another embodiment, R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂.

In another embodiment,
R¹ is C₁-C₄ alkyl;
R² is fluoro;
R³ is optionally substituted and is quinoline, quinoxaline, isoquinoline, thiazole, phenyl, naphthalene, thiophene, pyrrole, or pyridine; and
R⁴ is H or methyl.

In one embodiment, the compound of general formula (I) is:
{3-[1-(4-Chloro-phenyl)-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
[5-Fluoro-2-methyl-3-(1-naphthalen-2-yl-ethyl)-indol-1-yl]-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)-2-methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-1-yl]-acetic acid;
[5-Fluoro-2-methyl-3-(1,3-thiazol-2-ylmethyl)indol-1-yl]-acetic acid;
[3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-yl)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-({1-[(4-methylbenzene)sulfonyl]pyrrol-2-yl}methyl)indol-1-yl]-acetic acid;
[3-({1-[(2,4-Difluorobenzene)sulfonyl]pyrrol-2-yl}methyl)-5-fluoro-2-methylindol-1-yl]-acetic acid;
(3-{[2-(Benzenesulfonyl)phenyl]methyl}-5-fluoro-2-methylindol-1-yl)-acetic acid;
[3-({2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{[2-(Benzenesulfonyl)pyridin-3-yl]methyl}-5-fluoro-2-methyl indol-1-yl)-acetic acid;
[5-Fluoro-3-({2-[(4-fluorobenzene)sulfonyl]pyridin-3-yl}methyl)-2-methylindol-1-yl]-acetic acid;
[3-({2-[(4-Chlorobenzene)sulfonyl]pyridin-3-yl}methyl)-5-fluoro-2-methylindol-1-yl]-acetic acid;
2-(3-(4-(Benzylsulfonyl)benzyl)-5-fluoro-2-methyl-indol-1-yl)-acetic acid;
2-(3-(4-(4-Chlorobenzylsulfonyl)benzyl)-5-fluoro-2-methyl-indol-1-yl)-acetic acid;
2-(3-(3-(Benzylsulfonyl)benzyl)-5-fluoro-2-methyl-indol-1-yl)-acetic acid;
2-(5-Fluoro-3-(3-(4-fiuorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopentylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-1-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
{5-Fluoro-3-[(1-methanesulfonylnaphthalen-2-yl)methyl]-2-methylindol-1-yl}-acetic acid;
{5-Fluoro-3-[(6-methanesulfonylnaphthalen-2-yl)methyl]-2-methylindol-1-yl}-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]-acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-znethylindol-1-yl}-acetic acid;
(5-Fluoro-2-methyl-3-{pyrazolo[1,5-a]pyridin-3-ylmethyl}indol-1-yl)-acetic acid;
(5-Fluoro-3-{imidazo[1,2-a]pyridin-2-ylmethyl}-2-methylindol-1-yl)-acetic acid;
(5-Fluoro-2-methyl-3-{[2-(methylsulfanyl)phenyl]methyl}indol-1-yl)-acetic acid;
(5-Fluoro-2-methyl-3-{[3-(methylsulfanyl)phenyl]methyl}indol-1-yl)-acetic acid;
(5-Fluoro-2-methyl-3-{[4-(ethylsulfanyl)phenyl]methyl}indol-1-yl)-acetic acid;
(3-{[4-(Ethylsulfanyl)phenyl]methyl}-5-fluoro-2-methylindol-1-yl)-acetic acid;
(5-Fluoro-2-methyl-3-{[4-(n-propylsulfanyl)phenyl]methyl}indol-1-yl)-acetic acid;
(5-Fluoro-2-methyl-3-{[4-(i-propylsulfanyl)phenyl]methyl}indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-{[4-(pentan-3-ylsulfanyl)phenyl]methyl}indol-1-yl)-acetic acid;
[3-({4-[(Cyclopropylmethyl)sulfanyl]phenyl}methyl) 5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-{[2-(Ethanesulfonyl)phenyl]methyl}-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-{[(2-(Butane-2-sulfonyl)phenyl]methyl}-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-{[2-(pentane-1-sulfonyl)phenyl]methyl}indol-1-yl)-acetic acid;
(3-{[2-(Cyclopropylmethane)sulfonylphenyl]methyl}-5-fluoro-2-methylindol-1-yl)-acetic acid;
(5-Fluoro-2-methyl-3-{[2-(propylsulfamoyl)phenyl]methyl}indol-1-yl)-acetic acid;
(3-{[(2-(Butylsulfamnoyl)phenyl]methyl}-5-fluoro-2-methylindol-1-yl)-acetic acid;
(5-Fluoro-2-methyl-3-{[3-(propylsulfamoyl)phenyl]methyl}indol-1-yl)-acetic acid;
(3-{[3-(Butylsulfamoyl)phenyl]methyl}-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl}indol-1-yl)-acetic acid;
(5-Fluoro-2-methyl-3-{[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-{[4-(pentan-3-ylsulfonyl)phenyl]methyl}indol-1-yl)-acetic acid;
[3-({4-[(Cyclopropylmethyl)sulfonyl]phenyl}methyl)-5-fluoro-2-methylindol-1-yl]-acetic acid;
(5-Fluoro-2-methyl-3-{[4-(propylsulfamoyl)phenyl]methyl}indol-1-yl)-acetic acid;
(3-{[4-(Butylsulfamoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methylindol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1′-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1-yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzenesulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]-acetic acid;
{5-Fluoro-2-methyl-3-[(2-phenylphenyl)methyl]indol-1-yl}-acetic acid;
(3-{[1-(Benzenesulfonyl)indol-2-yl]methyl}-5-fluoro-2-methylindol-1-yl)-acetic acid;
(3-{[2-(4-Chlorophenyl)phenyl]methyl}-5-fluoro-2-methylindol-1-yl)-acetic acid;
(5-Fluoro-2-methyl-3-{[2-(4-methylphenyl)phenyl]methyl}indol-1-yl)-acetic acid;
{5-Fluoro-2-methyl-3-[(3-phenoxyphenyl)methyl]indol-1-yl}-acetic acid;
[5-Fluoro-3-({4-[(4-fluorophenyl)carbonyl]-1-methylpyrrol-2-yl}methyl)-2-methylindol-1-yl]-acetic acid;
{5-Fluoro-2-methyl-3-[(6-{[3-(trifluoromethyl)phenyl]methyl}pyridin-3-yl)methyl]indol-1-yl}-acetic acid;
{5-Fluoro-2-methyl-3-[(3-phenoxythiophen-2-yl)methyl]indol-1-yl}-acetic acid;
(3-{[2-(Benzenesulfonyl)-1,3-thiazol-5-yl]methyl}-5-fluoro-2-methylindol-1-yl)-acetic acid;
{3-[(1-Benzylpyrazol-4-yl)methyl]-5-fluoro-2-methylindol-1-yl}-acetic acid;
[3-{(5-(4-Chlorophenoxy)-1-methyl-3-(trifluoromethyl)pyrazol-4-yl]methyl}-5-fluoro-2-methylindol-1-yl)-acetic acid;
[3-({5-[(4-Chlorobenzene)sulfonyl]furan-2-yl}methyl)-5-fluoro-2-methylindol-1-yl]-acetic acid;
[3-({5-[(4-Chlorobenzene)sulfonyl]thiophen-2-yl}methyl)-5-fluoro-2-methylindol-1-yl]-acetic acid;
[3-({3-[(4-Chlorobenzene)sulfonyl]thiophen-2-y}methyl)-5-fluoro-2-methylindol-1-yl]-acetic acid;
{3-[(2-Benzylphenyl)methyl]-5-fluoro-2-methylindol-1-yl}-acetic acid;
or the C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂ esters of any of the above; wherein
   m is 1 or 2;
   n is 1-4;
   X is OR⁷ or N(R⁷)₂;
   R⁷ is hydrogen or methyl; and
   R⁸ is C₁-C₁₈ alkyl.

In one embodiment, the PPI is selected from the group consisting of omeprazole, esomeprazole, lansoprazole, dexlansoprazole, pantoprazole, and rabeprazole, or a pharmaceutically acceptable salt thereof.

In another embodiment, the CRTH2 antagonist is (5-fluoro-2-methyl-3-quinolin-2-ylmethyl-indol-1-yl)-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof.

In another embodiment, the CRTH2 antagonist is [5-fluoro-3-(4-methanesulfonyl-benzyl)-2-methyl-indol-1-yl]-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof.

In another embodiment, the CRTH2 antagonist is (3-{[2-(benzenesulfonyl)pyridin-3-yl]methyl}-5-fluoro-2-methylindol-1-yl)-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof.

In another embodiment, the CRTH2 antagonist is [5-fluoro-3-({2-[(4-fluorobenzene)sulfonyl]pyridin-3-yl}methyl)-2-methylindol-1-yl]-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof.

In another embodiment, the CRTH2 antagonist is 5-(acetylamino)-3-[(4-chlorophenyl)thio]-2-methyl-1H-indole-1-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof.

In another embodiment, the effects of the at least one CRTH2 antagonist and the at least one proton pump inhibitor are synergistic.

Another aspect of the invention is to provide a method of preventing, treating, or ameliorating eosinophilic esophagitis (EoE) in an individual, comprising administering to the individual a therapeutically effective amount of at least one CRTH2 antagonist and at least one proton pump inhibitor (PPI) and further administering at least one corticosteroid. In one embodiment, the corticosteroid is selected from the group consisting of hydrocortisone, dexamethasone, methylprednisolone, and prednisolone.

Another aspect of the invention is to provide a method of preventing, treating, or ameliorating eosinophilic esophagitis (EoE) in an individual, comprising administering to the individual a therapeutically effective amount of at least one CRTH2 antagonist and at least one proton pump inhibitor (PPI) and further administering an anti-IL-3 monoclonal antibody.

Another aspect of the invention is to provide a method of preventing, treating, or ameliorating eosinophilic esophagitis (EoE) in an individual, comprising administering to the individual a therapeutically effective amount of at least one CRTH2 antagonist and at least one proton pump inhibitor (PPI) and further administering montelukast.

Another aspect of the invention is a kit for the treatment of eosinophilic esophagitis comprising: (a) at least one CRTH2 antagonist; and (b) at least one proton pump inhibitor; wherein the kit is packaged in one or more suitable containers. In one embodiment, the one or more suitable containers is a blister pack.

Another aspect of the invention provides a method for the maintenance therapy of eosinophilic esophagitis comprising:
(a) firstly administering to an individual in need of such treatment a therapeutically effect amount of a corticosteroid for a first predetermined period of time; and
(b) subsequently administering to the individual a therapeutically effective amount of at least one CRTH2 antagonist or a pharmaceutically acceptable salt thereof and at least one proton pump inhibitor or a pharmaceutically acceptable salt thereof for a second predetermined period of time.

### BRIEF DESCRIPTION OF THE FIGURES

FIG. 1 is a bar graph showing the difference in % change in esophageal eosinophil load in proximal and distal biopsies compared to placebo for patients treated with the CRTH2 antagonist (5-fluoro-2-methyl-3-quinolin-2-ylmethyl-indol-1-yl)-acetic acid.

FIG. 2 is a bar graph comparing the % change in esophageal eosinophil load in patients receiving (5-fluoro-2-methyl-3-quinolin-2-ylmethyl-indol-1-yl)-acetic acid and esomeprazole, (5-fluoro-2-methyl-3-quinolin-2-ylmethyl-indol-1-yl)-acetic acid alone, esomeprazole alone, or a placebo.

### DETAILED DESCRIPTION OF THE INVENTION

The invention provides methods and compositions for preventing, treating, or ameliorating eosinophilic esophagitis (EoE) in an individual, comprising administering to the individual a therapeutically effective amount of at least one CRTH2 antagonist and at least one proton pump inhibitor (PPI). The invention also provides compositions comprising a CRTH2 antagonist and/or a PPI for use in preventing, treating, or ameliorating EoE in an individual.

EoE is characterised by an allergic response with involvement of mast cells and Th2 cells, in addition to eosinophils. The number of IgE-bearing mast cells is elevated in EoE tissue and examination of the mast cell transcriptome in such tissue has demonstrated the presence of mast cell products such as carboxpeptidase A3 and tryptase (Abonia et al., J. Allergy Clin. Immunol. 126:140-149 (2010)). The Th2 cell-derived cytokines interleukin 4, 5, and 13 are also elevated in EoE tissue (Blanchard et al., J. Allergy Clin. Immunol. 127:208-217 (2011)). Factors produced by activated mast cells and Th2 cells exposed to antigens in esophageal tissue are likely to be important in promoting tissue eosinophilia and other aspects of disease pathology. Prostaglandin D2 (PGD2) is one such product that is produced in high concentrations by mast cells and Th2 cells in response to immunological activation (Pettipher, Br. J. Pharmacol. 153 Suppl. 1:S191-S199 (2008)) and causes activation of Th2 cells, eosinophils, and basophils through a high affinity interaction with the G protein coupled receptor CRTH2 (chemoattractant receptor-homologous molecule expressed on Th2 cells—also known as DP2) (Hirai et al., J. Exp. Med. 193:255-261 (2001)). Mast cell-dependent activation of Th2 cells promoting enhanced migration and cytokine production is mediated by PGD2 acting on CRTH2 (Oyles et al., Immunology 119.362-368 (2006); Xue et al., Clin. Exp. Immunol. 156:126-133 (2009)). Paracrine activation of Th2 cells is also inhibited by CRTH2 antagonists (Vinall et al., Immunology 121:577-584 (2007)). Studies in animal models indicate that genetic ablation of CRTH2 or administration of CRTH2 antagonists is effective in reducing eosinophil and lymphocyte accumulation and Th2 cytokine production in response to allergen in sensitised airways and skin (Pettipher, 2008).

Consequently, it is proposed that PGD2 produced by mast cells in response to food allergens or airborne allergens will contribute to eosinophil accumulation and disease pathology in EoE.

In one embodiment, the CRTH2 antagonists are disclosed in U.S. Published Application No. 2011/0124683 and have general formula (I):

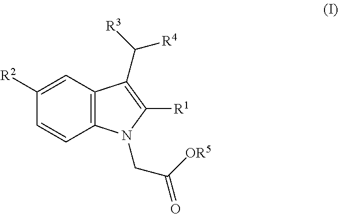

wherein

R¹ is C₁-C₆ alkyl;

R² is halogen;

R³ is aryl or heteroaryl optionally substituted with one or more substituents selected from halo, OH, CN, R⁶, COR⁶, CH₂R⁶, OR⁶, SR⁶, SO₂R⁶, or SO₂YR⁶;
R⁶ is C₁-C₆ alkyl, C₃-C₈ cycloalkyl, heterocyclyl, aryl, or heteroaryl, any of which may optionally be substituted with one or more substituents selected from halo, OH, CN, NO₂, C₁-C₆ alkyl, or O(C₁-C₆ alkyl); and
Y is NH or a straight or branched C₁-C₄ alkylene chain;

R⁴ is H or C₁-C₄ alkyl; and

R⁵ is hydrogen, C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R)₂;
m is 1 or 2;
n is 1-4;
X is OR⁷ or N(R⁷)₂;
R⁷ is hydrogen or methyl; and
R⁸ is C₁-C₁₈ alkyl;

or a pharmaceutically acceptable salt, hydrate, solvate, or complex thereof. See also U.S. Pat. Nos. 7,582,672, 7,750,027, 7,999,119, and 8,044,088, and U.S. published application Nos. 2009/0192195 and 2010/0022613.

In one embodiment of the invention, the compound of general formula (I) is a CRTH2 antagonist in which R⁵ is hydrogen.

In an alternative embodiment of the invention, the compound of general formula (I) is a prodrug for a CRTH2 antagonist and R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂; where
m is 1 or 2;
n is 1-4;
X is OR⁷ or N(R⁷)₂;
R⁷ is hydrogen or methyl; and
R⁸ is C₁-C₁₈ alkyl.

In one embodiment, the compound of general formula (I) is, independently or in any combination:
R¹ is C₁-C₄ alkyl, particularly methyl or ethyl but more especially methyl;
R² is fluoro;
R⁴ is H or methyl; and
R³ is quinoline, quinoxaline, isoquinoline, thiazole, phenyl, naphthalene, thiophene, pyrrole, or pyridine, any of which may optionally be substituted as set out above.

In another embodiment, R⁴ of formula (I) is H.

In one embodiment, R³ of formula (I) is optionally substituted quinoline, phenyl, naphthalene, thiophene, pyrrole, or pyridine.

In another embodiment, when R³ is quinoline or isoquinoline, it is suitably unsubstituted or substituted with one or more halo substituents, especially fluoro.

In one embodiment, when R³ is pyridyl, it is a 3-pyridyl moiety.

In another embodiment, when R³ is phenyl, naphthalene, thiophene, pyrrole, or pyridine, it may optionally have one or more substituents, with particularly suitable substituents including OR⁶, SO₂R⁶, or SO₂YR⁶; where R⁶ and Y are as defined above.

In one embodiment, R⁶ of formula (I) is C₁-C₆ alkyl, a 4- to 6-membered cycloalkyl group, a 5- or 6-membered heterocyclyl group, or phenyl, any of which may be substituted as defined above.

In one embodiment, Y, when present, is a CH₂ moiety.

In another embodiment, when R³ is substituted with SO₂R⁶ or SO₂YR⁶, the R⁶ group is generally unsubstituted or substituted with one or more substituents chosen from methyl and halo, particularly chloro or fluoro.

In another embodiment, when R³ is substituted with OR⁶, the R⁶ group may be unsubstituted or substituted with one or more substituents chosen from halo, cyano, C₁-C₄ alkyl, and O(C₁-C₄ alkyl).

Particular examples of compounds of formula (I) include:
{3-[1-(4-Chloro-phenyl)-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
[5-Fluoro-2-methyl-3-(1-naphthalen-2-yl-ethyl)-indol-1-yl]-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-yl]-acetic acid;
[5-Fluoro-2-methyl-3-(1,3-thiazol-2-ylmethyl)indol-1-yl]-acetic acid;
[3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-yl)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-({1-[(4-methylbenzene)sulfonyl]pyrrol-2-yl}methyl)indol-1-yl]-acetic acid;
[3-({1-[(2,4-Difluorobenzene)sulfonyl]pyrrol-2-yl}methyl)-5-fluoro-2-methylindol-1-yl]-acetic acid;
(3-{[2-(Benzenesulfonyl)phenyl]methyl}-5-fluoro-2-methylindol-1-yl)-acetic acid;
[3-({2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{[2-(Benzenesulfonyl)pyridin-3-yl]methyl}-5-fluoro-2-methylindol-1-yl)-acetic acid;
[5-Fluoro-3-({2-[(4-fluorobenzene)sulfonyl]pyridin-3-yl}methyl)-2-methylindol-1-yl]-acetic acid;
[3-({2-[(4-Chlorobenzene)sulfonyl]pyridin-3-yl}methyl)-5-fluoro-2-methylindol-1-yl]-acetic acid;
2-(3-(4-(Benzylsulfonyl)benzyl)-5-fluoro-2-methyl-indol-1-yl)-acetic acid,
2-(3-(4-(4-Chlorobenzylsulfonyl)benzyl)-5-fluoro-2-methyl-indol-1-yl)-acetic acid;
2-(3-(3-(Benzylsulfonyl)benzyl)-5-fluoro-2-methyl-indol-1-yl)-acetic acid;
2-(5-Fluoro-3-(3-(4-fluorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopentylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-2-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
{5-Fluoro-3-[(1-methanesulfonylnaphthalen-2-yl)methyl]-2-methylindol-1-yl}-acetic acid;
{5-Fluoro-3-[(6-methanesulfonylnaphthalen-2-yl)methyl]-2-methylindol-1-yl}-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]-acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-methylindol-1-yl}-acetic acid;
(5-Fluoro-2-methyl-3-{pyrazolo[1,5-a]pyridin-3-ylmethyl}indol-1-yl)-acetic acid;
(5-Fluoro-3-{imidazo[1,2-a]pyridin-2-ylmethyl}-2-methylindol-1-yl)-acetic acid;
(5-Fluoro-2-methyl-3-{[2-(methylsulfanyl)phenyl]methyl}indol-1-yl)-acetic acid;
(5-Fluoro-2-methyl-3-{[3-(methylsulfanyl)phenyl]methyl}indol-1-yl)-acetic acid;
(5-Fluoro-2-methyl-3-{[4-(ethylsulfanyl)phenyl]methyl}indol-1-yl)-acetic acid;
(3-{[4-(Ethylsulfanyl)phenyl]methyl}-5-fluoro-2-methylindol-1-yl)-acetic acid;
(5-Fluoro-2-methyl-3-{[4-(n-propylsulfanyl)phenyl]methyl}indol-1-yl)-acetic acid;
(5-Fluoro-2-methyl-3-{[4-(i-propylsulfanyl)phenyl]methyl}indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-{[4-(pentan-3-ylsulfanyl)phenyl]methyl}indol-1-yl)-acetic acid;
[3-({4-[(Cyclopropylmethyl)sulfanyl]phenyl}methyl)-5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-{[2-(Ethanesulfonyl)phenyl]methyl}-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-{[2-(Butane-2-sulfonyl)phenyl]methyl}-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-{[2-(pentane-1-sulfonyl)phenyl]methyl}indol-1-yl)-acetic acid;
(3-{[2-(Cyclopropylmethane)sulfonylphenyl]methyl}-5-fluoro-2-methylindol-1-yl)-acetic acid;
(5-Fluoro-2-methyl-3-{[2-(propylsulfamoyl)phenyl]methyl}indol-1-yl)-acetic acid;
(3-{[2-(Butylsulfamoyl)phenyl]methyl}-5-fluoro-2-methylindol-1-yl)-acetic acid;
(5-Fluoro-2-methyl-3-{[3-(propylsulfamnoyl)phenyl]methyl}indol-1-yl)-acetic acid;
(3-{[3-(Butylsulfamoyl)phenyl]methyl}-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl}indol-1-yl)-acetic acid;
(5-Fluoro-2-methyl-3-{[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-{[4-(pentan-3-ylsulfonyl)phenyl]methyl}indol-1-yl)-acetic acid;
[3-({4-[(Cyclopropylmethyl)sulfonyl]phenyl}methyl)-5-fluoro-2-methylindol-1-yl]-acetic acid;
(5-Fluoro-2-methyl-3-{[4-(propylsulfamoyl)phenyl]methyl}indol-1-yl)-acetic acid;
(3-{[4-(Butylsulfamnoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methyl indol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzenesulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]-acetic acid;
{5-Fluoro-2-methyl-3-[(2-phenylphenyl)methyl]indol-1-yl}-acetic acid;
(3-{[1-(Benzenesulfonyl)indol-2-yl]methyl}-5-fluoro-2-methylindol-1-yl)-acetic acid;
(3-{[2-(4-Chlorophenyl)phenyl]methyl}-5-fluoro-2-methylindol-1-yl)-acetic acid;
(5-Fluoro-2-methyl-3-{[2-(4-methylphenyl)phenyl]methyl}indol-1-yl)-acetic acid;
{5-Fluoro-2-methyl-3-[(3-phenoxyphenyl)methyl]indol-1-yl}-acetic acid;
[5-Fluoro-3-({4-[(4-fluorophenyl)carbonyl]-1-methylpyrrol-2-yl}methyl)-2-methylindol-1-yl]-acetic acid;
{5-Fluoro-2-methyl-3-[(6-{[3-(trifluoromethyl)phenyl]methyl}pyridin-3-yl)methyl]indol-1-yl}-acetic acid;
{5-Fluoro-2-methyl-3-[(3-phenoxythiophen-2-yl)methyl]indol-1-yl}-acetic acid;
(3-{[2-(Benzenesulfonyl)-1,3-thiazol-5-yl]methyl}-5-fluoro-2-methylindol-1-yl)-acetic acid;
{3-[(1-Benzylpyrazol-4-yl)methyl]-5-fluoro-2-methylindol-1-yl}-acetic acid;
(3-{[5-(4-Chlorophenoxy)-1-methyl-3-(trifluoromethyl)pyrazol-4-yl]methyl}-5-fluoro-2-methylindol-1-yl)-acetic acid;
[3-({5-[(4-Chlorobenzene)sulfonyl]furan-2-yl}methyl)-5-fluoro-2-methylindol-1-yl]-acetic acid;
[3-({5-[(4-Chlorobenzene)sulfonyl]thiophen-2-yl}methyl)-5-fluoro-2-methylindol-1-yl]-acetic acid;
[3-({3-[(4-Chlorobenzene)sulfonyl]thiophen-2-yl}methyl)-5-fluoro-2-methylindol-1-yl]-acetic acid;
{3-[(2-Benzylphenyl)methyl]-5-fluoro-2-methylindol-1-yl}-acetic acid;

or the C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂ esters of any of the above; wherein
m is 1 or 2;
n is 1-4;
X is OR⁷ or N(R⁷)₂;
R⁷ is hydrogen or methyl; and
R⁸ is C₁-C₁₈ alkyl.

The compounds of general formula (I) in which R⁵ is hydrogen are active as CRTH2 antagonists.

Prodrugs are any covalently bonded compounds which release the active parent drug according to general formula (I) in vivo. Examples of prodrugs include the compounds of general formula (I) in which R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂ or CH((CH₂)ₘO(C═O)R⁸)₂; where
m is 1 or 2;
n is 1-4;
X is OR⁷ or N(R⁷)₂;
R⁷ is hydrogen or methyl; and
R⁸ is C₁-C₁₈ alkyl.

Other CRTH2 antagonists which may be used in the practice of the invention include those disclosed in U.S. Pat. No. 7,754,735 having Formula (II):

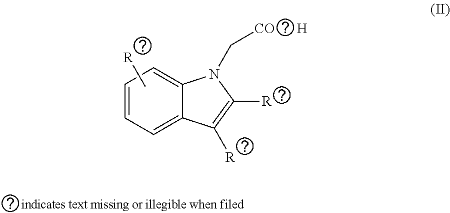

and pharmaceutically acceptable salts or solvates thereof, in which in which R¹ is hydrogen, halogen, CN, nitro, SO₂R⁴, OH, OR⁴, SR⁴, SOR⁴, SO₂NR⁵R⁶, CONR⁵R⁶, NR⁵R⁶, NR⁹SO₂R⁴, NR⁹CO₂R⁴, NR⁹COR⁴, heteroaryl, aryl (optionally substituted by chlorine or fluorine), C₂-C₆ alkenyl, C₂-C₆ alkynyl or C₁-C₆ alkyl, the latter three groups being optionally substituted by one or more substituents independently selected from halogen, OR⁸ and NR⁵R⁶, S(O)ₓR⁷ where x is 0, 1 or 2; R² is hydrogen, halogen, CN, SO₂R⁴ or CONR⁵R⁶, CH₂OH, CH₂OR⁴ or C₁₋₇alkyl, the latter group being optionally substituted by one or more substituents independently selected from halogen atoms, OR⁸ and NR⁵R⁶, S(O)ₓR⁷ where x is 0, 1 or 2; R³ is quinoline, 1,2-benzisothiazole, benzo[b]thiophene or indole each of which is optionally substituted by one or more substituents independently selected from hydrogen, halogen, CN, nitro, OH, SO₂R⁴, OR⁴, SR⁴, SOR⁴, SO₂NR⁵R⁶, CONR⁵R⁶, NR⁵R⁶, NR⁹SO₂R⁴, NR⁹CO₂R⁴, NR⁹CO₂H, NR⁹COR⁴, C₂-C₆ alkenyl, C₂-C₆ alkynyl, C₁₋₆ alkyl, the latter three groups being optionally substituted by one or more substituents independently selected from halogen atoms, OR⁸ and NR⁵R⁶, S(O)ₓR⁷ where x=0, 1 or 2; R⁴ represents aryl, heteroaryl, or C₁-6alkyl all of which may be optionally substituted by one or more substituents independently selected from halogen atoms, aryl, heteroaryl, OR¹⁰ and NR¹¹R¹², S(O)ₓR¹³ (where x=0, 1 or 2), CONR¹⁴R¹⁵, NR¹⁴COR¹⁵, SO₂NR¹⁴R¹⁵, NR¹⁴SO₂R¹⁵; R⁵ and R⁶ independently represent a hydrogen atom, a C₁₋₆ alkyl group, or an aryl, or a heteroaryl, the latter three of which may be optionally substituted by one or more substituents independently selected from halogen atoms, aryl, OR⁸ and NR¹⁴R¹⁵, CONR¹⁴R¹⁵, NR¹⁴COR¹⁵, SO₂NR¹⁴R¹⁵, NR¹⁴SO₂R¹⁵; or R⁵ and R⁶ together with the nitrogen atom to which they are attached can form a 3-8 membered saturated heterocyclic ring optionally containing one or more atoms selected from O, S(O)ₓ where x=0, 1 or 2, NR¹⁶, and itself optionally substituted by C₁₋₃ alkyl; R⁷ and R¹³ independently represent a C₁-C₆, alkyl, an aryl or a heteroaryl group all of which maybe optionally substituted by one or more halogen atoms; R⁸ represents a hydrogen atom, C(O)R⁹, C₁-C₆ alkyl an aryl or a heteroaryl group, all of which may be optionally substituted by halogen atoms or an aryl group; each of R⁹, R¹⁰, R¹¹, R¹², R¹⁴, and R¹⁵, independently represents a hydrogen atom, C₁-C₆ alkyl, an aryl or a heteroaryl group, all of which may be optionally substituted by a halogen atom; and R¹⁶ is hydrogen, C₁₋₄ alkyl, —COC₁-C₄ alkyl, COYC₁-C₄alkyl where Y is O or NR⁷.

Examples of compounds of Formula (II) include 3-(2-chloro-4-quinolinyl)-2,5-dimethyl-1H-indole-1-acetic acid; 3-(2-chloro-4-quinolinyl)-2-methyl-1H-indole-1-acetic acid; 3-(2-chloro-4-quinolinyl)-1H-indole-1-acetic acid; 2-methyl-3-(4-quinolinyl)-1H-indole-1-acetic acid; 3-(2-chloro-4-quinolinyl)-5-methoxy-2-methyl-1H-indole-1-acetic acid; 3-(2-chloro-4-quinolinyl)-2,6-dimethyl-1H-indole-1-acetic acid; 3-(2-chloro-4-quinolinyl)-2,4-dimethyl-1H-indole-1-acetic acid; 2,5-dimethyl-3-(7-methyl-4-quinolinyl)-1H-indole-1-acetic acid; 2,5-dimethyl-3-(8-methyl-4-quinolinyl)-1H-indole-1-acetic acid; 3-(6-fluoro-4-quinolinyl)-2,5-dimethyl-1H-indole-1-acetic acid; 3-(6-methoxy-4-quinolinyl)-2,5-dimethyl-1H-indole-1-acetic acid; 2,5-dimethyl-3-(4-quinolinyl)-1H-indole-1-acetic acid; 2,5-dimethyl-3-[8-(trifluoromethyl)-4-quinolinyl]-1H-indole-1-acetic acid; 3-(7-chloro-4-quinolinyl)-2,5-dimethyl-6-(methylsulfonyl)-1H-indole-1-acetic acid; 3-(8-fluoro-4-quinolinyl)-2,5-dimethyl-1H-indole-1-acetic acid; 3-(2,8-dimethyl-4-quinolinyl)-2,5-dimethyl-1H-indole-1-acetic acid; 2,5-dimethyl-3-[7-(trifluoromethyl)-4-quinolinyl]-1H-indole-1-acetic acid; 3-(8-bromo-2-methyl-4-quinolinyl)-2,5-dimethyl-1H-indole-1-acetic acid; 3-(8-methoxy-2-methyl-4-quinolinyl)-2,5-dimethyl-1H-indole-1-acetic acid; 3-(6,8-dimethyl-4-quinolinyl)-2,5-dimethyl-1H-indole-1-acetic acid; 3-(8-chloro-4-quinolinyl)-2,5-dimethyl-1H-indole-1-acetic acid; 3-(7-chloro-4-quinolinyl)-2-methyl-5-nitro-1H-indole-1-acetic acid; 5-chloro-3-(7-chloro-4-quinolinyl)-2-methyl-1H-indole-1-acetic acid; 5-chloro-2-methyl-3-(8-methyl-4-quinolinyl)-1H-indole-1-acetic acid; 5-chloro-3-(6-methoxy-2-methyl-4-quinolinyl)-2-methyl-1H-indole-1-acetic acid; 5-methoxy-2-methyl-3-(8-methyl-4-quinolinyl)-1H-indole-1-acetic acid, sodium salt; 3-(7-chloro-4-quinolinyl)-5-fluoro-2-methyl-1H-indole-1-acetic acid; 5-fluoro-2-methyl-3-[8-(trifluoromethyl)-4-quinolinyl]-1H-indole-1-acetic acid; 5-fluoro-2-methyl-3-(8-methyl-4-quinolinyl)-1H-indole-1-acetic acid; 2-methyl-3-(8-methyl-4-quinolinyl)-5-(trifluoromethyl)-1H-indole-1-acetic acid; 3-(8-nitroquinolin-4-yl)-2,5-dimethyl-1H-indole-1-acetic acid; 3-(S-cyano-4-quinolinyl)-2,5-dimethyl-1H-indole-1-acetic acid; 2,5-dimethyl-3-[8-(methylsulfonyl)-4-quinolinyl]-1H-indole-1-acetic acid; 3-[8-(difluoromethoxy)-4-quinolinyl]-2,5-dimethyl-1H-indole-1-acetic acid; 5-amino-3-(7-chloro-4-quinolinyl)-2-methyl-1H-indole-1-acetic acid; 3-(7-chloro-4-quinolinyl)-2-methyl-5-[(methylsulfonyl)amino]-1H-indole-1-acetic acid; 5-(acetylamino)-3-(7-chloro-4-quinolinyl)-2-methyl-1H-indole-1-acetic acid; 3-(7-chloro-4-quinolin-4-yl)-5-fluoro-2,4-dimethyl-1H-indol-1-yl]acetic acid; 5-chloro-2-methyl-3-(8-quinolinyl)-1H-indole-1-acetic acid; 5-chloro-3-(7-chloro-4-quinolinyl)-2-(hydroxymethyl)-1H-indole-1-acetic acid; 5-chloro-3-(7-chloro-4-quinolinyl)-2-(methoxymethyl)-1H-indole-1-acetic acid; 2-[(acetyloxy)methyl]-5-chloro-3-(7-chloro-4-quinolinyl)-1H-indole-1-acetic acid; 5-chloro-3-(7-chloro-4-quinolinyl)-2-[(methylamino)methyl]-1H-indole-1-acetic acid; 5-chloro-3-(7-chloro-4-quinolinyl)-2-[(methylthio)methyl]-1H-indole-1-acetic acid; 5-chloro-3-(7-chloro-4-quinolinyl)-2-[(methylsulfonyl)methyl]-1H-indole-1-acetic acid; 3-(7-chloro-4-quinoliny)-4-methoxy-2-methyl-1H-indole-1-acetic acid; 5-chloro-2-methyl-3-[8-(trifluoromethyl)-4-quinolinyl]-1H-indole-1-acetic acid; 5-cyano-2-methyl-3-(8-methyl-4-quinolinyl)-1H-indole-1-acetic acid; 5-cyano-2-methyl-3-[8-(trifluoromethyl)-4-quinolinyl]-1H-indole-1-acetic acid; 3-(7-chloro-4-quinolinyl)-5-cyano-2-methyl-1H-indole-1-acetic acid; 3-(8-chloro-4-quinolinyl)-5-cyano-2-methyl-1H-indole-1-acetic acid; 5-cyano-2-methyl-3-(2-methyl-4-quinolinyl)-1H-indole-1-acetic acid; 3-(8-chloro-4-quinolinyl)-5-fluoro-2-methyl-1H-indole-1-acetic acid; 5-fluoro-2-methyl-3-(7-methyl-4-quinolinyl)-1H-indole-1-acetic acid; 2-methyl-5-(trifluoromethyl)-3-[8-(trifluoromethyl)-4-quinolinyl]-1H-indole-1-acetic acid; 3-(8-fluoro-4-quinolinyl)-2-methyl-5-(trifluoromethyl)-1H-indole-1-acetic acid; 3-(8-chloro-4-quinolinyl)-2-methyl-5-(trifluoromethyl)-1H-indole-1-acetic acid; 3-(8-chloro-4-quinolinyl)-2-methyl-5-(methylsulfonyl)-1H-indole-1-acetic acid; 2-methyl-3-(8-methyl-4-quinolinyl)-5-(methylsulfonyl)-1H-indole-1-acetic acid; 2-methyl-5-(methylsulfonyl)-3-[8-(trifluoromethyl)-4-quinolinyl]-1H-indole-1-acetic acid; 3-(7-chloro-4-quinolinyl)-2-methyl-5-(methylsulfonyl)-1H-indole-1-acetic acid; 5-chloro-2-methyl-3-[8-(methylsulfonyl)-4-quinolinyl]-1H-indole-1-acetic acid; and 5-fluoro-2-methyl-3-[8-(methylsulfonyl)-4-quinolinyl]-1H-indole-1-acetic acid.

Other CRTH2 antagonists which may be used in the practice of the invention include those disclosed in U.S. Pat. No. 7,723,373 having Formula (III):

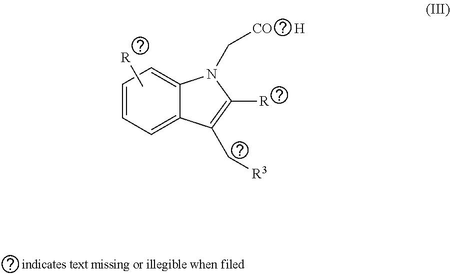

and pharmaceutically acceptable salts thereof, in which: n represents 1 or 2; R¹ is one or more substituents independently selected from halogen, CN, nitro, SO₂R⁴, OR⁴, SR⁴, SOR⁴, SO₂NR⁵R⁶, CONR⁵R⁶, NR⁵R⁶, NR⁵SO₂R⁴, NR⁹CO₂R⁴, NR⁹COR⁴, aryl, heteroaryl, C₂-C₆ alkenyl, C₂-C₆ alkynyl or C₁₋₆ alkyl, the latter five groups being optionally substituted by one or more substituents independently selected from halogen, OR⁷ and NR⁸R⁹, NR⁸R⁹, S(O)ₓR⁷ where x is 0, 1 or 2; R² is hydrogen, halogen, CN, SO₂R⁴ or CONR⁵R⁶, COR⁴ or C₁₋₇ alkyl, the latter group being optionally substituted by one or more substituents independently selected from halogen atoms, OR⁸ and NR⁵R⁶, S(O)ₓR⁷ where x is 0, 1 or 2; R³ is aryl or a 5-7 membered heteroaryl ring containing one or more heteratoms selected from N, S and O, each of which is optionally substituted by one or more substituents independently selected from halogen, CN, nitro, SO₂R⁴, OH, OR⁴, SR⁴, SOR⁴, SO₂NR⁵R⁶, CONR⁵R⁶, NR⁵R⁶, NR⁹SO₂R⁴, NR⁹CO₂R⁴, NR⁹COR⁴, C₂-C₆ alkenyl, C₂-C₆ alkynyl, C₁-C₆ alkyl, the latter three groups being optionally substituted by one or more substituents independently selected from halogen atoms, OR⁷ and NR⁸R⁹, S(O)ₓR⁷ where x is 0, 1 or 2; R⁴ represents aryl, heteroaryl, or C₁-C₆ alkyl, all of which may be optionally substituted by one or more substituents independently selected from halogen atoms, aryl, heteroaryl, OR¹⁰ and NR¹¹R¹²S(O)ₓR¹³ (where x=0, 1 or 2), CONR¹⁴R⁵, NR¹⁴COR¹⁵, SO₂NR¹⁴R¹⁵, NR¹⁴SO₂R¹⁵, CN, nitro; R⁵ and R⁶ independently represent a hydrogen atom, a C₁-C₆ alkyl group, an aryl, or a heteroaryl, the latter three of which may be optionally substituted by one or more substituents independently selected from halogen atoms, aryl, OR¹³ and NR¹⁴R¹⁵, CONR¹⁴R¹⁵, NR¹⁴COR¹⁵, SO₂NR¹⁴R¹⁵, NR¹⁴SO₂R¹⁵, CN, nitro; or R⁵ and R⁶ together with the nitrogen atom to which they are attached can form a 3-8 membered saturated heterocyclic ring optionally containing one or more atoms selected from O, S(O), where x is 0, 1 or 2, NR¹⁶, and the ring itself optionally substituted by C₁-C₃ alkyl; R⁷ and R¹³ independently represent a C₁-C₆ alkyl group, an aryl or heteroaryl group all of which may be optionally substituted by halogen atoms; R¹ represents a hydrogen atom, C(O)R⁹, C₁-C₆ alkyl (optionally substituted by halogen atoms, aryl or heteroaryl groups, both of which may also be optionally substituted by one or more fluorine atoms); an aryl or a heteroaryl group, which may be optionally substituted by one or more halogen atoms; each of R⁹, R¹⁰, R¹¹, R¹², R¹⁴, R¹⁵, independently represents a hydrogen atom, C₁-C₆ alkyl, an aryl or a heteroaryl group (all of which may be optionally substituted by one or more halogen atoms); and R¹⁶ is hydrogen, C₁₋₄ alkyl, —C(O)C₁-C₄ alkyl, C(O)YC₁-C₄alkyl, Y is O or NR⁷.

Examples of compounds having Formula (III) include 3-[(4-chlorophenyl)sulfonyl]-2,5-dimethyl-1H-indol-1-acetic acid; 5-chloro-3-[(4-chlorophenyl)sulfonyl]-2-methyl-1H-indole-1-acetic acid; 6-chloro-3-[(4-chlorophenyl)sulfonyl]-2-methyl-1H-indole-1-acetic acid; 7-chloro-3-[(4-chlorophenyl)sulfonyl]-2-methyl-1H-indole-1-acetic acid; 5-chloro-3-[(4-chlorophenyl)sulfonyl]-4-cyano-2-methyl-1H-indole-1-acetic acid; 5-chloro-3-[(4-chlorophenyl)sulfonyl]-6-cyano-2-methyl-1H-indole-1-acetic acid; 3-[(4-chlorophenyl)sulfonyl]-2,5-dimethyl-1H-indole-1-acetic acid; 3-[(4-chlorophenyl)sulfonyl]-4-(ethylsulfonyl)-7-methoxy-2-methyl-1-H-indole-1-acetic acid; 3-[(4-chlorophenyl)sulfonyl]-5-cyano-2-methyl-1H-indole-1-acetic acid; 3-[(4-chlorophenyl)sulfonyl]-5-cyano-2-methyl-1H-indole-1-acetic acid; 5-chloro-3-[(4-chlorophenyl)sulfonyl]-2-methyl-1H-indole-1-acetic acid, 4-chloro-3-[(4-chlorophenyl)sulfonyl]-2-methyl-1H-indole-1-acetic acid; 3-[(4-methoxyphenyl)sulfonyl]-2,5-dimethyl-1H-indol-1-acetic acid; 3-[(3-methoxyphenyl)sulfonyl]-2,5-dimethyl-1H-indol-1-acetic acid; 3-[(2-Chlorophenyl)sulfonyl]-2,5-dimethyl-1H-indol-1-acetic acid; 3-[(3-Chlorophenyl)sulfonyl]-2,5-dimethyl-1H-indol-1-acetic acid; 3-[(4-Cyanophenyl)sulfonyl]-2,5-dimethyl-1H-indole-1-acetic acid; 3-[(2-methylphenyl)sulfonyl]-2,5-Dimethyl-1H-indol-1-acetic acid; 3-[(2-ethylphenyl)sulfonyl]-2,5-dimethyl-1H-indol-1-acetic acid; 3-[(4-chlorophenyl)sulfonyl]-2-methyl-4-nitro-1H-indole-1-acetic acid; 4-(Acetylamino)-3-[(4-chlorophenyl)sulfonyl]-2-methyl-1H-indole-1-acetic acid; 3-[(4-chlorophenyl)sulfonyl]-2-methyl-4-[(methylsulfonyl)amino]-1H-indole-1-acetic acid; 3-[(4-chlorophenyl)sulfonyl]-4-(ethylamino)-2-methyl-1H-indole-1-acetic acid; 3-[(2,6-Dichlorophenyl)sulfonyl]-2,5-dimethyl-1H-indole-1-acetic acid; 3-[(4-chlorophenyl)sulfonyl]-2-methyl-4-phenyl-1H-indole-1-acetic acid 3-[(4-chlorophenyl)sulfonyl]-5-fluoro-2-methyl-1H-indole-1-acetic acid, 3-[(3-chlorophenyl)sulfonyl]-5-fluoro-2-methyl-1H-indole-1-acetic acid, and 5-fluoro-2-methyl-3-[[4-(trifluoromethyl)phenyl]sulfonyl]-1H-indole-1-acetic acid.

Other CRTH2 antagonists which may be used in the practice of the invention include those disclosed in U.S. Pat. No. 7,687,535 having Formula (IV):

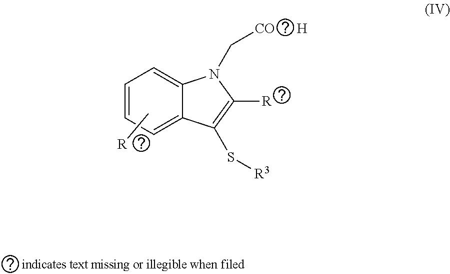

and pharmaceutically acceptable salts thereof, in which: R¹ is one or more substituents independently selected from NR⁴SO₂R⁵, NR⁴CO₂R⁶, NR⁴COR⁶, NR⁴SO₂NR⁵R⁶, NHSO₂R⁵, NHCO₂R⁶, NHCOR⁶, NHCONR⁴, NHSO₂NR⁵R⁶, or heteroaryl, the latter which may be optionally substituted by halogen, CN, OR⁷, C₁₋₃ alkyl (which may be optionally substituted by halogen atoms); R² is hydrogen, halogen, CN, SO₂R⁴ or CONR⁵R⁶, CH₂OH, CH₂OR⁴ or C₁₋₇ alkyl, the latter group being optionally substituted by one or more substituents independently selected from halogen atoms, OR⁸ and NR⁵R⁶, S(O)ₓR⁷ where x is 0, 1 or 2; R³ is aryl or heteroaryl each of which is optionally substituted by one or more substituents independently selected from hydrogen, halogen, CN, OH, SO₂R⁴, OR⁴, SR⁴, SOR⁴, SO₂NR⁵R⁶, CONR⁵R⁶, NR⁵R⁶, NHSO₂R⁴, NHCOR⁴, NHCO₂R⁴, NR⁷SO₂R⁴, NR⁷CO₂R⁴, NR⁷COR⁴, C₂-C₆ alkenyl, C₂-C₆ alkynyl, C₁₋₆ alkyl, the latter three groups being optionally substituted by one or more substituents independently selected from halogen atoms, OR⁸ and NR⁵R⁶, S(O)ₓR⁷ where x is 0, 1 or 2; R⁴ represents aryl, heteroaryl, or C₁₋₆ alkyl, all of which may be optionally substituted by one or more substituents independently selected from halogen atoms, aryl, heteroaryl, OR¹⁰, OH, NR¹¹R¹², S(O)ₓR¹³ (where x is 0, 1 or 2), CONR¹⁴R¹⁵, NR¹⁴COR¹⁵, SO₂NR¹⁴R¹⁵, NR¹⁴SO₂R¹⁵, CN, nitro; R⁵ and R⁶ independently represent a hydrogen atom, a C₁₋₆ alkyl group, or an aryl, or a heteroaryl, the latter three of which may be optionally substituted by one or more substituents independently selected from halogen atoms, aryl, OR⁸ and NR¹⁴R¹⁵, CONR¹⁴R¹⁵, NR¹⁴COR¹⁵, SO₂NR¹⁴R¹⁵, NR¹⁴SO₂R¹⁵; CN, nitro, C₁₋₃ alkyl (which may be optionally substituted by halogen atoms); or R⁵ and R⁶ together with the nitrogen atom to which they are attached can form a 3-8 membered saturated heterocyclic ring optionally containing one or more atoms selected from O, S(O)ₓ where x is 0, 1 or 2, NR¹⁶, and itself optionally substituted by C₁₋₃ alkyl; R⁷ and R¹³ independently represent a C₁-C₆ alkyl, an aryl or a heteroaryl group, all of which may be optionally substituted by halogen atoms; R⁸ represents a hydrogen atom, C(O)R⁹, C₁-C₆ alkyl (optionally substituted by halogen atoms or aryl) an aryl or a heteroaryl group (optionally substituted by halogen); each of R⁹, R¹⁰, R¹¹, R¹², R¹⁴, R¹⁵, independently represents a hydrogen atom, C₁-C₆ alkyl, an aryl or a heteroaryl group (all of which may be optionally substituted by halogen atoms); and R¹⁶ is hydrogen, C₁₋₄ alkyl, COC₁-C₄ alkyl or COYC₁-C₄alkyl where Y is O or NR⁷.

Examples of compounds having Formula (IV) include 4-(acetylamino)-3-[(4-chlorophenyl)thio]-2-methyl-1H-indole-1-acetic acid; 3-[(4-chlorophenyl)thio]-2-methyl-4-[(methylsulfonyl)amino]-1H-indole-1-acetic acid; 3-[(4-chlorophenyl)thio]-2-methyl-4-(5-pyrimidinyl)-1H-indole-1-acetic acid; 3-[(4-chlorophenyl)thio]-2-methyl-4-pyrazinyl-1H-indole-1-acetic acid; 3-[(2-chlorophenyl)thio]-2-methyl-5-[(methylsulfonyl)amino]-1H-indole-1-acetic acid; 3-[(3-chlorophenyl)thio]-2-methyl-4-[(methylsulfonyl)amino]-1H-indole-1-acetic acid; 3-[(4-chlorophenyl)thio]-2-methyl-4-[(methylsulfonyl)amino]-1H-indole-1-acetic acid; 3-[(3-methoxyphenyl)thio]-2-methyl-4-[(methylsulfonyl)amino]-1H-indole-1-acetic acid; 3-[(4-methoxyphenyl)thio]-2-methyl-4-[(methylsulfonyl)amino]-1H-indole-1-acetic acid; 3-[(2-trifluoromethylphenyl)thio]-2-methyl-4-[(methylsulfonyl)amino]-1H-indole-1-acetic acid; 3-[(8-Quinolinyl)thio]-2-methyl-4-[(methylsulfonyl)amino]-1H-indole-1-acetic acid; 3-[(2-(methylethyl)phenyl)thio]-2-methyl-4-[(methylsulfonyl)amino]-1H-indole-1-acetic acid; 5-(acetylamino)-3-[(4-chlorophenyl)thio]-2-methyl-1H-indole-1-acetic acid; 4-(acetylethylamino)-3-[(4-chlorophenyl)thio]-2-methyl-1H-indole-1-acetic acid; 3-[(4-chlorophenyl)thio]-4-[cyclopropylcarbonyl)amino]-2-methyl-1H-indole-1-acetic acid; 4-(benzoylamino)-3-[(4-chlorophenyl)thio]-2-methyl-1H-indole-1-acetic acid; 4-(acetylamino)-3-[(3-chlorophenyl)thio]-2-methyl-1H-indole-1-acetic acid; 3-[(4-chlorophenyl)thio]-4-[[(dimethylamino)sulfonyl]amino]-2-methyl-1H-indole-1-acetic acid; 3-[(4-chlorophenyl)thio]-2-methyl-4-[[(1-methyl-1H-imidazol-4-yl)sulfonyl]amino]-1H-indole-1-acetic acid; 3-[(4-chlorophenyl)thio]-4-[[(dimethylamino)acetyl]amino]-2-methyl-1H-indole-1-acetic acid; 4-(acetylamino)-2-methyl-3-[[4-(methylsulfonyl)phenyl]thio]-1H-indole-1-acetic acid; 4-(acetylamino)-3-[(2-chlorophenyl)thio]-2-methyl-1H-indole-1-acetic acid; 4-(acetylamino)-2-methyl-3-[[4-(ethylsulfonyl)phenyl]thio]-1H-indole-1-acetic acid; 3-[(4-chlorophenyl)thio]-4-[[(ethylamino)carbonyl]amino]-2-methyl-1H-indole-1-acetic acid; 3-[[4-(methylsulfonyl)phenyl]thio]-4-(5-pyrimidinyl)-1H-indole-1-acetic acid 2-methyl-3-[[4-(methylsulfonyl)phenyl]thio]-4-(2-thiophenyl)-1H-indole-1-acetic acid 4-(3,5-dimethyl-4-isoxazolyl)-2-methyl-3-[[4-(methylsulfonyl)phenyl]thio]-1H-indole-1-acetic acid 4-(3-furanyl)-2-methyl-3-[[4-(methylsulfonyl)phenyl]thio]-1H-indole-1-acetic acid 2-methyl-4-[(methylsulfonyl)amino]-3-[[4-(methylsulfonyl)phenyl]thio]-1H-indole-1-acetic acid, 2-methyl-5-[(methylsulfonyl)amino]-3-[[3-(methylsulfonyl)phenyl]thio]-1H-indole-1-acetic acid, 2-methyl-5-[(methylsulfonyl)amino]-3-[[2-(methylsulfonyl)phenyl]thio]-1H-indole-1-acetic acid, 2-methyl-3-[[4-(methylsulfonyl)phenyl]thio]-5-(5-pyrimidinyl)-1H-indole-1-acetic acid, 2-methyl-3-[[4-(methylsulfonyl)phenyl]thio]-5-(3-thiophenyl)-1H-indole-1-acetic acid, 5-(3,5-dimethyl-4-isoxazolyl)-2-methyl-3-[[4-(methylsulfonyl)phenyl]thio]-1H-indole-1-acetic acid, 2-methyl-3-[[4-(methylsulfonyl)phenyl]thio]-5-(3-pyridinyl)-1H-indole-1-acetic acid, 2-methyl-3-[[4-(methylsulfonyl)phenyl]thio]-5-(1H-pyrazolyl)-1H-indole-1-acetic acid, and 4-(acetylamino)-3-[(4-cyanophenyl)thio]-2-methyl-1H-indole-1-acetic acid.

Other CRTH2 antagonists which may be used in the practice of the invention include those disclosed in U.S. Pat. No. 7,709,521 having Formula (V):

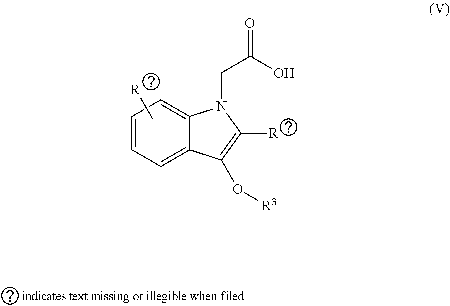

and pharmaceutically acceptable salts or solvates thereof, wherein R¹ is one or more substituents selected from hydrogen, halogen, CN, nitro, SO₂R⁴, OH, OR⁴, S(O)ₓR⁴, SO₂N⁵R⁶, CONR⁵R⁶, NR⁵R⁶, NR⁹SO₂R⁴, NR⁹SO₂NR⁵R⁶, NR⁹CO₂R⁴, NR⁹COR⁴, aryl, heteroaryl, C₂-C₆ alkenyl, C₂-C₆ alkynyl or C₁₋₆ alkyl the latter five groups being optionally substituted by one or more substituents independently selected from halogen, CN, NR⁹SO₂R⁴, NR⁹CO₂R⁴, NR⁹COR⁴, OR⁸ and NR⁵R⁶, S(O)ₓR⁷ where x is 0, 1 or 2; R² is hydrogen, halogen, CN, SO₂R⁴ or CONR⁵R⁶, CH₂OH, CH₂OR⁴ or C₁₋₇ alkyl, the latter group being optionally substituted by one or more substituents independently selected from halogen atoms, OR⁸ and NR⁵R⁶, S(O)ₓR⁷ where x is 0, 1 or 2; R³ is aryl or heteroaryl each of which is optionally substituted by one or more substituents independently selected from hydrogen, halogen, CN, nitro, OH, SO₂R⁴, OR⁴, SR⁴, SOR⁴, SO₂NR⁵R⁶, CONR⁵R⁶, NR⁵R⁶, NHSO₂R⁴, NHCO₂R⁴, NHCOR⁴, NR⁷SO₂R⁴, NR⁷CO₂R⁴, NR⁷COR⁴, NHC₁₋₆alkylNR⁵R⁶, C₂-C₆ alkenyl, C₂-C₆ alkynyl, C₁₋₆ alkyl, the latter three groups being optionally substituted by one or more substituents independently selected from halogen atoms, CN, OR⁸ and NR⁵R⁶, S(O)ₓR⁷ where x=0, 1 or 2; R⁴ represents aryl, heteroaryl, or C₁₋₆ alkyl all of which may be optionally substituted by one or more substituents independently selected from halogen atoms, aryl, heteroaryl, OR¹⁰, OH, NR¹¹R¹², S(O)ₓR¹³ (where x=0, 1 or 2), CONR¹⁴R¹⁵, NR¹⁴COR¹⁵, SO₂NR¹⁴R¹⁵, NR¹⁴SO₂R¹⁵, CN, nitro; R⁵ and R⁶ independently represent a hydrogen atom, a C₁₋₆ alkyl group, or an aryl, or a heteroaryl, the latter three of which may be optionally substituted by one or more substituents independently selected from halogen atoms, aryl, OR⁸ and NR¹⁴R¹⁵, CONR¹⁴R¹⁵, NR¹⁴COR¹⁵, SO₂NR¹⁴R¹⁵, NR¹⁴SO₂R¹⁵; CN, nitro, or R⁵ and R⁶ together with the nitrogen atom to which they are attached can form a 3-8 membered saturated heterocyclic ring optionally containing one or more atoms selected from O, S(O)ₓ where x=0, 1 or 2, NR¹⁶, and itself optionally substituted by C₁₋₃ alkyl; R⁷ and R¹³ independently represent a C₁-C₆ alkyl, an aryl or a heteroaryl group, all of which may be optionally substituted by halogen atoms; R⁸ represents a hydrogen atom, C(O)R⁹, C₁-C₆ alkyl (optionally substituted by halogen atoms or aryl) an aryl or a heteroaryl group (optionally substituted by halogen); each of R⁹, R¹⁰, R¹¹, R¹², R¹⁴, R¹⁵, independently represents a hydrogen atom, C₁-C₆ alkyl, an aryl or a heteroaryl group (all of which may be optionally substituted by halogen atoms); and R¹⁶ is hydrogen, C₁₋₄ alkyl, —COC₁-C₄ alkyl, COYC₁-C₄alkyl where Y is O or NR⁷.

Examples of compounds having Formula (V) include 3-(4-Chlorophenoxy)-5-fluoro-2-methyl-1H-indole-1-acetic acid; 5-Fluoro-2-methyl-3-[4-(methylsulfonyl)phenoxy]-1H-indole-1-acetic acid; 3-(4-Chlorophenoxy)-2-methyl-4-[(methylsulfonyl)amino]-1H-indole-1-acetic acid; 4-(Acetylamino)-3-(4-chlorophenoxy)-2-methyl-1H-indole-1-acetic acid; 3-(4-chlorophenoxy)-2-methyl-5-(methylsulfonyl)-1H-indole-1-acetic acid; 3-(4-chlorophenoxy)-2-methyl-5-(trifluoromethyl) 1H-indole-1-acetic acid; 3-(4-Chlorophenoxy)-2-methyl-5-[(methylsulfonyl)amino]-1H-indole-1-acetic acid; 3-(4-Chlorophenoxy)-5-[(ethylsulfonyl)amino]-2-methyl 1H-indole-1-acetic acid; 3-(4-Carboxyphenoxy)-5-fluoro-2-methyl-1H-indole-1-acetic acid; 5-Fluoro-2-methyl-3-[4-[(methylamino)carbonyl]phenoxy]-1H-indole-1-acetic acid; 3-[4-[(Ethylamino)carbonyl]phenoxy]-5-fluoro-2-methyl-1H-indole-1-acetic acid; 5-Fluoro-2-methyl-3-[4-[[(1-methylethyl)amino]carbonyl]phenoxy]-1H-indole-1-acetic acid; 3-(4-Carboxyphenoxy)-5-chloro-2-methyl-1H-indole-1-acetic acid; 5-Fluoro-3-[4-(methoxycarbonyl)phenoxy]-2-methyl-1H-indole-1-acetic acid; 5-Chloro-3-[4-(methoxycarbonyl)phenoxy]-2-methyl-1H-indole-1-acetic acid; 5-Chloro-2-methyl-3-[4-[(methylamino)carbonyl]phenoxy]-1H-indole-1-acetic acid; 5-Chloro-3-[4-[(ethylamino)carbonyl]phenoxy]-2-methyl-1H-indole-1-acetic acid; Sodium 5-Chloro-2-methyl-3-[4-[[(1-methylethyl)amino]carbonyl]phenoxy]-1H-indole-1-acetate; 3-[4-[[(2-Aminoethyl)amino]carbonyl]phenoxy]-5-fluoro-2-methyl-1H-indole-1-acetic acid; 2,5-Dimethyl-3-[4-(methylsulfonyl)phenoxy]-1H-indole-1-acetic acid; 2-Methyl-3-[4-(methylsulfonyl)phenoxy]-5-(trifluoromethyl) 1H-indole-1-acetic acid; 5-Chloro-α,2-dimethyl-3-[4-(methylsulfonyl)phenoxy]-1H-indole-1-acetic acid; 5-Cyano-2-methyl-3-[4-(methylsulfonyl)phenoxy]-1H-indole-1-acetic acid; 3-(4-Chlorophenoxy)-4-[(ethylsulfonyl)amino]-2-methyl 1H-indole-1-acetic acid; 3-(4-Chlorophenoxy)-4-[[(dimethylamino)sulfonyl]amino]-2-methyl-1H-indole-1-acetic acid; 3-(4-Chlorophenoxy)-2-methyl-4-pyrazinyl-1H-indole-1-acetic acid; 3-(4-Chlorophenoxy)-2-methyl-4-[[(1-methylethyl)sulfonyl]amino]-1H-indole-1-acetic acid; 3-[4-[(Dimethylamino)sulfonyl]phenoxy]-5-fluoro-2-methyl-1H-indole-1-acetic acid; 3-[4-(Ethylsulfonyl)phenoxy]-5-fluoro-2-methyl-1H-indole-1-acetic acid; 3-[4-(Ethylsulfonyl)phenoxy]-2-methyl-5-(trifluoromethyl)-1H-indole-1-acetic acid; 3-(4-Cyanophenoxy)-2-methyl-5-(trifluoromethyl)-1H-indole-1-acetic acid; and 3-(4-Cyanophenoxy)-5-fluoro-2-methyl-1H-indole-1-acetic acid.

Other CRTH2 antagonists which may be used in the practice of the invention include those disclosed in U.S. Pat. No. 7,714,132 having Formula (VI):

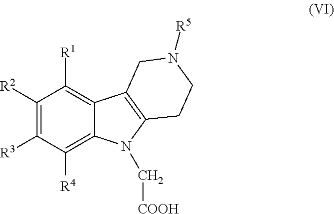

wherein R¹, R², R³ and R⁴ independently represent hydrogen, C₁-C₅-alkyl, C₁-C₅-alkoxy, halogen, nitro, cyano or formyl; and R⁵ represents C₀-C₅-alkyl-carbonyl, C₂-C₅-alkenyl-carbonyl, C₁-C₅-alkoxy-carbonyl, C₁-C₅-alkyl, C₁-C₅-alkyl-carbamoyl, aryl-C₁-C₅-alkyl, aryl-carbonyl, aryl-C₁-C₅-alkyl-carbonyl, aryl-C₁-C₅-alkoxy-carbonyl, aryl-carbamoyl, aryl-thiocarbamoyl, aryl-C₁-C₅-alkyl-carbamoyl, aryl-C₁-C₅-alkyl-thiocarbamoyl, cycloalkyl-carbonyl, cycloalkyl-C₁-C₅-alkyl-carbonyl, cycloalkyl-C₁-C₅-alkoxy-carbonyl, cycloalkyl-carbamoyl, heteroaryl-C₁-C₅-alkyl, heteroaryl-carbonyl, heteroaryl-C₁-C₅-alkyl-carbonyl or heteroaryl-C₁-C₅-alkoxy-carbonyl; with the proviso that when R¹, R², R³ and R⁴ represent hydrogen, R⁵ is not an ethoxy-carbonyl group or a tert-butoxycarbonyl group; and optically pure enantiomers, mixtures of enantiomers, racemates, optically pure diastereoisomers, mixtures of diastereoisomers, diastercoisomeric racemates, mixtures of diastereoisomeric racemates, meso forms, and salts thereof.

Examples of compounds having Formula (VI) include: (2-benzyloxycarbonyl-1,2,3,4-tetrahydro-pyrido[4,3-b]indol-5-yl)-acetic acid; (2-butoxycarbonyl-1,2,3,4-tetrahydro-pyrido[4,3-b]indol-5-yl)-acetic acid; (2-9H-fluoren-9-ylmethoxycarbonyl-1,2,3,4-tetrahydro-pyrido[4,3-b]-indol-5-yl)-acetic acid; (2-acetyl-1,2,3,4-tetrahydro-pyrido[4,3-b]indol-5-yl)-acetic acid; (2-phenylacetyl-1,2,3,4-tetrahydro-pyrido[4,3-b]indol-5-yl)-acetic acid; (2-benzoyl-1,2,3,4-tetrahydro-pyrido[4,3-b]indol-5-yl)-acetic acid; [2-(3,4,5-trimethoxy-benzoyl)-1,2,3,4-tetrahydro-pyrido[4,3-b]indol-5-yl]-acetic acid; (2-cyclohexanecarbonyl-1,2,3,4-tetrahydro-pyrido[4,3-b]indol-5-yl)-acetic acid; [2-(4-methoxy-benzoyl)-1,2,3,4-tetrahydro-pyrido[4,3-b]indol-5-yl]-acetic acid; [2-(thiophene-2-carbonyl)-1,2,3,4-tetrahydro-pyrido[4,3-b]indol-5-yl]-acetic acid; [2-(furan-2-carbonyl)-1,2,3,4-tetrahydro-pyrido[4,3-b]indol-5-yl]-acetic acid; (2-cyclopropanecarbonyl-1,2,3,4-tetrahydro-pyrido[4,3-b]indol-5-yl)-acetic acid; [2-(naphthalene-1-carbonyl)-1,2,3,4-tetrahydro-pyrido[4,3-b]indol-5-yl]-acetic acid; [2-(2-methoxy-benzoyl)-1,2,3,4-tetrahydro-pyrido[4,3-b]indol-5-yl]-acetic acid; [2-(4-trifluoromethyl-benzoyl)-1,2,3,4-tetrahydro-pyrido[4,3-b]indol-5-yl]-acetic acid; [2-(3,5-bis-trifluoromethyl-benzoyl)-1,2,3,4-tetrahydro-pyrido[4,3-b]indol-5-yl]-acetic acid; [2-(3-cyclopentyl-propionyl)-1,2,3,4-tetrahydro-pyrido[4,3-b]indol-5-yl]-acetic acid; [2-(3-phenyl-propionyl)-1,2,3,4-tetrahydro-pyrido[4,3-b]indol-5-yl]-acetic acid; [2-(biphenyl-4-carbonyl)-1,2,3,4-tetrahydro-pyrido[4,3-b]indol-5-yl]-acetic acid; [2-(4-tert.-butyl-benzoyl)-1,2,3,4-tetrahydro-pyrido[4,3-b]indol-5-yl]-acetic acid; [2-(4-trifluoromethoxy-benzoyl)-1,2,3,4-tetrahydro-pyrido[4,3-b]indol-5-yl]-acetic acid; [2-((E)-but-2-enoyl)-1,2,3,4-tetrahydro-pyrido[4,3-b]indol-5-yl]-acetic acid; [2-(4-chloro-benzoyl)-1,2,3,4-tetrahydro-pyrido[4,3-b]indol-5-yl]-acetic acid; [2-(3,5-d methoxy-benzoyl)-1,2,3,4-tetrahydro-pyrido[4,3-b]indol-5-yl]-acetic acid; (2-diphenylacetyl-1,2,3,4-tetrahydro-pyrido[4,3-b]indol-5-yl)-acetic acid; (2-hexanoyl-1,2,3,4-tetrahydro-pyrido[4,3-b]indol-5-yl)-acetic acid; [2-(3-chloro-benzoyl)-1,2,3,4-tetrahydro-pyrido[4,3-b]indol-5-yl]-acetic acid; [2-(4-bromo-benzoyl)-1,2,3,4-tetrahydro-pyrido[4,3-b]indol-5-yl]-acetic acid; [2-(pyridine-3-carbonyl)-1,2,3,4-tetrahydro-pyrido[4,3-b]indol-5-yl-]-acetic acid; (2-benzoyl-8-methoxy-1,2,3,4-tetrahydro-pyrido[4,3-b]indol-5-yl)-acetic acid; (2-benzoyl-7-methyl-1,2,3,4-tetrahydro-pyrido[4,3-b]indol-5-yl)-acetic acid; (2-benzoyl-8-bromo-1,2,3,4-tetrahydro-pyrido[4,3-b]indol-5-yl)-acetic acid; (2-benzoyl-8-methyl-1,2,3,4-tetrahydro-pyrido[4,3-b]indol-5-yl)-acetic acid; (2-benzoyl-6-methyl-1,2,3,4-tetrahydro-pyrido[4,3-b]indol-5-yl)-acetic acid; [2-(pyrazine-2-carbonyl)-1,2,3,4-tetrahydro-pyrido[4,3-b]indol-5-yl]-acetic acid; [2-(2-bromo-3-methyl-benzoyl)-1,2,3,4-tetrahydro-pyrido[4,3-b]indol-5-yl]-acetic acid; [2-(4′-ethyl-biphenyl-4-carbonyl)-1,2,3,4-tetrahydro-pyrido[4,3-b]indol-5-yl]-acetic acid; [2-(2-bromo-5-methyl-benzoyl)-1,2,3,4-tetrahydro-pyrido[4,3-b]indol-5-yl]-acetic acid; [2-(2-chloro-6-methyl-pyridine-4-carbonyl)-1,2,3,4-tetrahydro-pyrido[4,3-b]indol-5-yl]-acetic acid; [2-(biphenyl-2-carbonyl)-1,2,3,4-tetrahydro-pyrido[4,3-b]indol-5-yl]-acetic acid; [2-(5-bromo-furan-2-carbonyl)-1,2,3,4-tetrahydro-pyrido[4,3-b]indol-5-yl]-acetic acid; [2-(3-methyl-furan-2-carbonyl)-1,2,3,4-tetrahydro-pyrido[4,3-b]indol-5-yl]-acetic acid; [2-(2-methyl-furan-3-carbonyl)-1,2,3,4-tetrahydro-pyrido[4,3-b]indol-5-yl]-acetic acid; [2-(benzo[b]thiophene-2-carbonyl)-1,2,3,4-tetrahydro-pyrido[4,3-b]indol-5-yl]-acetic acid; [2-(5-chloro-thiophene-2-carbonyl)-1,2,3,4-tetrahydro-pyrido[4,3-b]indol-5-yl]-acetic acid; [2-(furan-3-carbonyl)-1,2,3,4-tetrahydro-pyrido[4,3-b]indol-5-yl]-acetic acid; [2-(2-naphthalen-2-yl-acetyl)-1,2,3,4-tetrahydro-pyrido[4,3-b]indol-5-yl]-acetic acid; [2-(thiophene-3-carbonyl)-1,2,3,4-tetrahydro-pyrido[4,3-b]indol-5-yl]-acetic acid; [2-(2-naphthalen-1-yl-acetyl)-1,2,3,4-tetrahydro-pyrido[4,3-b]indol-5-yl]-acetic acid; rac. [2-(2-cyclohexyl-2-phenyl-acetyl)-1,2,3,4-tetrahydro-pyrido[4,3-b]indol-5-yl]-acetic acid; (2-phenylcarbamoyl-1,2,3,4-tetrahydro-pyrido[4,3-b]indol-5-yl)-acetic acid; (2-ethylcarbamoyl-1,2,3,4-tetrahydro-pyrido[4,3-b]indol-5-yl)-acetic acid; sodium (2-phenethyl-1,2,3,4-tetrahydro-pyrido[4,3-b]indol-5-yl)-acetate; sodium [2-(3-phenyl-propyl)-1,2,3,4-tetrahydro-pyrido[4,3-b]indol-5-yl]-acetate; [2-(2-ethoxy-naphthalene-1-carbonyl)-1,2,3,4-tetrahydro-pyrido[4,3-b]indol-5-yl]-acetic acid; [2-(3-methyl-thiophene-2-carbonyl)-1,2,3,4-tetrahydro-pyrido[4,3-b]indol-5-yl]-acetic acid; [2-(5-methyl-thiophene-2-carbonyl)-1,2,3,4-tetrahydro-pyrido[4,3-b]indol-5-yl]-acetic acid; and [2-(pyridine-4-carbonyl)-1,2,3,4-tetrahydro-pyrido[4,3-b]indol-5-yl]-acetic acid.

In a more particular embodiment, the compound of general Formula (VI) is: [2-(naphthalene-1-carbonyl)-1,2,3,4-tetrahydro-pyrido[4,3-b]indol-5-yl]-acetic acid; [2-(3-chloro-benzoyl)-1,2,3,4-tetrahydro-pyrido[4,3-b]indol-5-yl]-acetic acid; [2-(4′-ethyl-biphenyl-4-carbonyl)-1,2,3,4-tetrahydro-pyrido[4,3-b]indol-5-yl]-acetic acid; [2-(2-bromo-3-methyl-benzoyl)-1,2,3,4-tetrahydro-pyrido[4,3-b]indol-5-yl]-acetic acid; (2-benzoyl-8-bromo-1,2,3,4-tetrahydro-pyrido[4,3-b]indol-5-yl)-acetic acid; (2-benzoyl-1,2,3,4-tetrahydro-pyrido[4,3-b]indol-5-yl)-acetic acid; [2-(4-bromo-benzoyl)-1,2,3,4-tetrahydro-pyrido[4,3-b]indol-5-yl]acetic acid; or [2-(furan-2-carbonyl)-1,2,3,4-tetrahydro-pyrido[4,3-b]indol-5-yl]acetic acid.

In a more particular embodiment, the compound of general Formula (VI) is selected from the group consisting of: 5-carboxymethyl-7-chloro-1,3,4,5-tetrahydro-pyrido[4,3-b]indole-2-carboxylic acid tert-butyl ester; 5-carboxymethyl-8-chloro-1,3,4,5-tetrahydro-pyrido[4,3-b]indole-2-carboxylic acid tert-butyl ester; 5-carboxymethyl-6-chloro-1,3,4,5-tetrahydro-pyrido[4,3-b]indole-2-carboxylic acid tert-butyl ester; 5-carboxymethyl-7-methyl-1,3,4,5-tetrahydro-pyrido[4,3-b]indole-2-carboxylic acid tert-butyl ester; 5-carboxymethyl-5-methyl-1,3,4,5-tetrahydro-pyrido[4,3-b]indole-2-carboxylic acid tert-butyl ester; 8-bromo-5-carboxymethyl-1,3,4,5-tetrahydro-pyrido[4,3-b]indole-2-carboxylic acid tert-butyl ester; 5-carboxymethyl-8-fluoro-1,3,4,5-tetrahydro-pyrido[4,3-b]indole-2-carboxylic acid tert-butyl ester; [7-chloro-2-(3-chloro-benzoyl)-1,2,3,4-tetrahydro-pyrido[4,3-b]indol-5-yl]-acetic acid; [8-chloro-2-(3-chloro-benzoyl)-1,2,3,4-tetrahydro-pyrido[4,3-b]indol-5-yl]-acetic acid; [6-chloro-2-(3-chloro-benzoyl)-1,2,3,4-tetrahydro-pyrido-[4,3-b]indol-5-yl]-acetic acid; [2-(3-chloro-benzoyl)-7-methyl-1,2,3,4-tetrahydro-pyrido[4,3-b]indol-5-yl]-acetic acid; [2-(3-chloro-benzoyl)-8-methyl-1,2,3,4-tetrahydro-pyrido[4,3-b]indol-5-yl]-acetic acid; [8-bromo-2-(3-chloro-benzoyl)-1,2,3,4-tetrahydro-pyrido[4,3-b]indol-5-yl]-acetic acid; [2-(3-chloro-benzoyl)-8-fluoro-1,2,3,4-tetrahydro pyrido[4,3-b]indol-5-yl]-acetic acid; [8-chloro-2-(thiophene-2-carbonyl)-1,2,3,4-tetrahydro-pyrido[4,3-b]indol-5-yl]-acetic acid; [6-chloro-2-(thiophene-2-carbonyl)-1,2,3,4-tetrahydro-pyrido[4,3-b]indol-5-yl]-acetic acid; [8-bromo-2-(thiophene-2-carbonyl)-1,2,3,4-tetrahydro-pyrido[4,3-b]indol-5-yl]-acetic acid; [8-fluoro-2-(thiophene-2-carbonyl)-1,2,3,4-tetrahydro-pyrido[4,3-b]indol-5-yl]-acetic acid; [7-chloro-2-(thiophene-2-carbonyl)-1,2,3,4-tetrahydro-pyrido[4,3-b]indol-5-yl]-acetic acid; [7-methyl-2-(thiophene-2-carbonyl)-1,2,3,4-tetrahydro-pyrido[4,3-b]indol-5-yl]-acetic acid; [8-methyl-2-(thiophene-2-carbonyl)-1,2,3,4-tetrahydro-pyrido[4,3-b]indol-5-yl]-acetic acid; [8-fluoro-2-(2-methoxy-naphthalene-1-carbonyl)-1,2,3,4-tetrahydro-pyrido[4,3-b]indol-5-yl]-acetic acid; [8-fluoro-2-(4-methoxy-naphthalene-1-carbonyl)-1,2,3,4-tetrahydro-pyrido[4,3-b]indol-5-yl]-acetic acid; [8-chloro-2-(2-methoxy-naphthalene-1-carbonyl)-1,2,3,4-tetrahydro-pyrido[4,3-b]indol-5-yl]-acetic acid; [8-chloro-2-(4-methoxy-naphthalene-1-carbonyl)-1,2,3,4-tetrahydro-pyrido[4,3-b]indol-5-yl]-acetic acid; [2-(2-methoxy-naphthalene-1-carbonyl)-8-methyl-1,2,3,4-tetrahydro-pyrido[4,3-b]indol-5-yl]-acetic acid; [2-(4-methoxy-naphthalene-1-carbonyl)-7-methyl-1,2,3,4-tetrahydro-pyrido[4,3-b]indol-5-yl]-acetic acid; [2-(2-methoxy-naphthalene-1-carbonyl)-7-methyl-1,2,3,4-tetrahydro-pyrido[4,3-b]indol-5-yl]-acetic acid; [2-(2-ethoxy-naphthalene-1-carbonyl)-8-methyl-1,2,3,4-tetrahydro-pyrido[4,3-b]indol-5-yl]-acetic acid; [2-(2-ethoxy-naphthalene-1-carbonyl)-7-methyl-1,2,3,4-tetrahydro-pyrido[4,3-b]indol-5-yl]-acetic acid; [2-(4-methoxy-naphthalene-1-carbonyl)-7-methyl-1,2,3,4-tetrahydro-pyrido[4,3-b]indol-5-yl]-acetic acid; [2-(2-fluoro-benzoyl)-1,2,3,4-tetrahydro-pyrido[4,3-b]indol-5-yl]-acetic acid; [2-(3-fluoro-benzoyl)-1,2,3,4-tetrahydro-pyrido[4,3-b]indol-5-yl]-acetic acid; [2-(3,5-difluoro-benzoyl)-1,2,3,4-tetrahydro-pyrido[4,3-b]indol-5-yl]-acetic acid; [2-(3,4,5-trifluoro-benzoyl)-1,2,3,4-tetrahydro-pyrido[4,3-b]indol-5-yl]-acetic acid; [2-(2,3,4,5-tetrafluoro-benzoyl)-1,2,3,4-tetrahydro-pyrido[4,3-b]indol-5-yl]-acetic acid; (2-benzoyl-8-fluoro-1,2,3,4-tetrahydro-pyrido[4,3-b]indol-5-yl)-acetic acid; (2-benzoyl-6-chloro-1,2,3,4-tetrahydro-pyrido[4,3-b]indol-5-yl)-acetic acid; (2-benzoyl-8-isopropyl-1,2,3,4-tetrahydro-pyrido[4,3-b]indol-5-yl)-acetic acid; (2-benzoyl-8-chloro-1,2,3,4-tetrahydro-pyrido[4,3-b]indol-5-yl)-acetic acid; (2-benzoyl-7,8-dichloro-1,2,3,4-tetrahydro-pyrido[4,3-b]indol-5-yl)-acetic acid; (2-benzoyl-8-trifluoromethyl-1,2,3,4-tetrahydro-pyrido[4,3-b]indol-5-yl)-acetic acid; (2-benzoyl-8-tert-butyl-1,2,3,4-tetrahydro-pyrido[4,3-b]indol-5-yl)-acetic acid; (2-benzoyl-7-chloro-8-methyl-1,2,3,4-tetrahydro-pyrido[4,3-b]indol-5-yl)-acetic acid; 5 (2-benzoyl-7,8-dimethyl-1,2,3,4-tetrahydro-pyrido[4,3-b]indol-5-yl)-acetic acid; (2-benzoyl-7-fluoro-1,2,3,4-tetrahydro-pyrido[4,3-b]indol-5-yl)-acetic acid; [7-chloro-2-(2-naphthalen-1-yl-acetyl)-1,2,3,4-tetrahydro-pyrido[4,3-b]indo-5-yl]I-acetic acid; [8-chloro-2-(2-naphthalen-1-yl-acetyl)-1,2,3,4-tetrahydro-pyrido[4,3-b]indol-5-yl]-acetic acid; [7-methyl-2-(2-naphthalen-1-yl-acetyl)-1,2,3,4-tetrahydro-pyrido[4,3-b]indol-5-yl]-acetic acid; [8-bromo-2-(2-naphthalen-1-yl-acetyl)-1,2,3,4-tetrahydro-pyrido[4,3-b]indol-5-yl]-acetic acid; [2-(4′-ethyl-biphenyl-4-carbonyl)-7-methyl-1,2,3,4-tetrahydro-pyrido[4,3-b]indol-5-yl]-acetic acid; [8-bromo-2-(4′-ethyl-biphenyl-4-carbonyl)-1,2,3,4-tetrahydro-pyrido[4,3-b]indo-5-yl]-acetic acid; [2-(4′-ethyl-biphenyl-4-carbonyl)-8-fluoro-1,2,3,4-tetrahydro-pyrido[4,3-b]indol-5-yl]-acetic acid; [6-chloro-2-(4′-ethyl-biphenyl-4-carbonyl)-1,2,3,4-tetrahydro-pyrido[4,3-b]indol-5-yl]-acetic acid; [7-chloro-2-(4′-ethyl-biphenyl-4-carbonyl)-1,2,3,4-tetrahydro-pyrido[4,3-b]indol-5-yl]-acetic acid; [8-chloro-2-(4′-ethyl-biphenyl-4-carbonyl)-1,2,3,4-tetrahydro-pyrido[4,3-b]indol-5-yl]-acetic acid; [2-(4′-ethyl-biphenyl-4-carbonyl)-8-methyl-1,2,3,4-tetrahydro-pyrido[4,3-b]indol-5-yl]-acetic acid; [8-methyl-2-(2-naphthalen-1-yl-acetyl)-1,2,3,4-tetrahydro-pyrido[4,3-b]indol-5-yl]-acetic acid; [6-chloro-2-(2-naphthalen-1-yl-acetyl)-1,2,3,4-tetrahydro-pyrido[4,3-b]indol-5-yl]-acetic acid; [8-chloro-2-(naphthalene-1-carbonyl)-1,2,3,4-tetrahydro-pyrido[4,3-b]indol-5-yl]-acetic acid; [6-chloro-2-(naphthalene-1-carbonyl)-1,2,3,4-tetrahydro-pyrido[4,3-b]indol-5-yl]-acetic acid; [7-methyl-2-(naphthalene-1-carbonyl)-1,2,3,4-tetrahydro-pyrido[4,3-b]indol-5-yl]-acetic acid; [8-methyl-2-(naphthalene-1-carbonyl)-1,2,3,4-tetrahydro-pyrido[4,3-b]indol-5-yl]-acetic acid; [8-bromo-2-(naphthalene-1-carbonyl)-1,2,3,4-tetrahydro-pyrido[4,3-b]indol-5-yl]-acetic acid; [8-fluoro-2-(naphthalene-1-carbonyl)-1,2,3,4-tetrahydro-pyrido[4,3-b]indol-5-yl]-acetic acid; [8-fluoro-2-(2-naphthalen-1-yl-acetyl)-1,2,3,4-tetrahydro-pyrido[4,3-b]indol-5-yl]-acetic acid; [2-(2-bromo-3-methyl-benzoyl)-7-chloro-1,2,3,4-tetrahydro-pyrido[4,3-b]indol-5-yl]-acetic acid; [2-(2-bromo-3-methyl-benzoyl)-8-chloro-1,2,3,4-tetrahydro-pyrido[4,3-b]indol-5-yl]-acetic acid; [2-(2-bromo-3-methyl-benzoyl)-6-chloro-1,2,3,4-tetrahydro-pyrido[4,3-b]indol-5-yl]-acetic acid; [2-(2-bromo-3-methyl-benzoyl)-7-methyl-1,2,3,4-tetrahydro-pyrido[4,3-b]indol-5-yl]-acetic acid; [2-(2-bromo-3-methyl-benzoyl)-8-methyl-1,2,3,4-tetrahydro-pyrido[4,3-b]indol-5-yl]-acetic acid; [8-bromo-2-(2-bromo-3-methyl-benzoyl)-1,2,3,4-tetrahydro-pyrido[4,3-b]indol-5-yl]-acetic acid; [2-(2-bromo-3-methyl-benzoyl)-8-fluoro-1,2,3,4-tetrahydro-pyrido[4,3-b]indol-5-yl]-acetic acid; [8-bromo-2-(2-ethoxy-naphthalene-1-carbonyl)-1,2,3,4-tetrahydro-pyrido[4,3-b]indol-5-yl]-acetic acid; [2-(2-ethoxy-naphthalene-1-carbonyl)-8-fluoro-1,2,3,4-tetrahydro-pyrido[4,3-b]indol-5-yl]-acetic acid; [8-chloro-2-(2-ethoxy-naphthalene-1-carbonyl)-1,2,3,4-tetrahydro-pyrido[4,3-b]indol-5-yl]-acetic acid; [2-(4-methoxy-naphthalene-1-carbonyl)-1,2,3,4-tetrahydro-pyrido[4,3-b]indol-5-yl]-acetic acid; [2-(5-bromo-naphthalene-1-carbonyl)-1,2,3,4-tetrahydro-pyrido[4,3-b]indol-5-yl]-acetic acid; [2-(4-methyl-naphthalene-1-carbonyl)-1,2,3,4-tetrahydro-pyrido[4,3-b]indol-5-yl]-acetic acid; [2-(2-methyl-naphthalene-1-carbonyl)-1,2,3,4-tetrahydro-pyrido[4,3-b]indol-5-yl]-acetic acid; [2-(biphenyl-3-carbonyl)-1,2,3,4-tetrahydro-pyrido[4,3-b]indol-5-yl]-acetic acid; [2-(4-fluoro-naphthalene-1-carbonyl)-1,2,3,4-tetrahydro-pyrido[4,3-b]indol-5-yl]-acetic acid; [2-(2-methoxy-naphthalene-1-carbonyl)-1,2,3,4-tetrahydro-pyrido[4,3-b]indol-5-yl]-acetic acid; 2-(9-oxo-9H-fluorene-2-carbonyl)-1,2,3,4-tetrahydro-pyrido[4,3-b]indol-5-yl]-acetic acid; (2-(9H-fluorene-1-carbonyl)-1,2,3,4-tetrahydro-pyrido[4,3-b]indol-5-yl)-acetic acid; [2-(9H-fluorene-4-carbonyl)-1,2,3,4-tetrahydro-pyrido[4,3-b]indol-5-yl]-acetic acid; [2-(2,4,6-trifluoro-benzoyl)-1,2,3,4-tetrahydro-pyrido[4,3-b]indol-5-yl]-acetic acid; [2-(4-cyclohexyl-benzoyl)-1,2,3,4-tetrahydro-pyrido[4,3-b]indol-5-yl]-acetic acid; [2-(1H-indole-4-carbonyl)-1,2,3,4-tetrahydro-pyrido[4,3-b]indol-5-yl]-acetic acid; [2-(2-fluoro-phenylcarbamoyl)-1,2,3,4-tetrahydro-pyrido[4,3-b]indol-5-yl]-acetic acid; [2-(3-fluoro-phenylcarbamoyl)-1,2,3,4-tetrahydro-pyrido[4,3-b]indol-5-yl]-acetic acid; [2-(4-fluoro-phenylcarbamoyl)-1,2,3,4-tetrahydro-pyrido[4,3-b]indol-5-yl]-acetic acid; (2-o-tolylcarbamoyl-1,2,3,4-tetrahydro-pyrido[4,3-b]indol-5-yl)-acetic acid; (2-m-tolylcarbamoyl-1,2,3,4-tetrahydro-pyrido[4,3-b]indol-5-yl)-acetic acid; (2-p-tolylcarbamoyl-1,2,3,4-tetrahydro-pyrido[4,3-b]indol-5-yl)-acetic acid; (2-benzylcarbamoyl-1,2,3,4-tetrahydro-pyrido[4,3-b]indol-5-yl)-acetic acid; (2-phenethylcarbamoyl-1,2,3,4-tetrahydro-pyrido[4,3-b]indol-5-yl)-acetic acid; [2-(naphthalen-1-ylcarbamoyl)-1,2,3,4-tetrahydro-pyrido[4,3-b]indol-5-yl]-acetic acid; [2-(naphthalen-2-ylcarbamoyl)-1,2,3,4-tetrahydro-pyrido[4,3-b]indol-5-yl]-acetic acid; [2-(biphenyl-2-ylcarbamoyl)-1,2,3,4-tetrahydro-pyrido[4,3-b]indol-5-yl]-acetic acid; (2-cyclohexylcarbamoyl-1,2,3,4-tetrahydro-pyrido[4,3-b]indol-5-yl)-acetic acid; [2-(2-chloro-phenylcarbamoyl)-1,2,3,4-tetrahydro-pyrido[4,3-b]indol-5-yl]-acetic acid; [2-(4-fluoro-phenylthiocarbamoyl)-1,2,3,4-tetrahydro-pyrido[4,3-b]indol-5-yl]-acetic acid; (2-phenylthiocarbamoyl-1,2,3,4-tetrahydro-pyrido[4,3-b]indol-5-yl)-acetic acid; (2-phenethylthiocarbamoyl-1,2,3,4-tetrahydro-pyrido[4,3-b]indol-5-yl)-acetic acid; (2-cyclohexylthiocarbamoyl-1,2,3,4-tetrahydro-pyrido[4,3-b]indol-5-yl)-acetic acid; (2-benzylthiocarbamoyl-1,2,3,4-tetrahydro-pyrido[4,3-b]indol-5-yl)-acetic acid; [2-(2-chloro-phenylthiocarbamoyl)-1,2,3,4-tetrahydro-pyrido[4,3-b]indol-5-yl]-acetic acid; (2-p-tolylthiocarbamoyl-1,2,3,4-tetrahydro-pyrido[4,3-b]indol-5-yl)-acetic acid; (2-m-tolylthiocarbamoyl-1,2,3,4-tetrahydro-pyrido[4,3-b]indol-5-yl)-acetic acid; and (2-o-tolylthiocarbamoyl-1,2,3,4-tetrahydro-pyrido[4,3-b]indol-5-yl)-acetic acid.

Other CRTH2 antagonists which may be used in the practice of the invention include those disclosed in U.S. Pat. Appl. Publication No. 2009/275659 having Formula (VII):

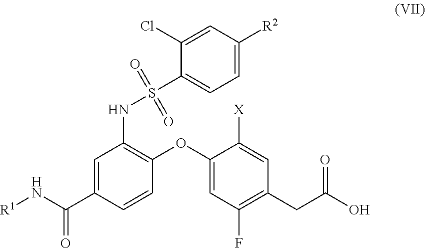

and salts thereof wherein R¹ is alkyl or cycloalkyl; R² is halo, alkyl, haloalkyl, alkoxy, haloalkoxy, or cycloalkyl; and X is chloro or fluoro. In a particular embodiment, the compound of Formula (VII) is [5-chloro-4-(2-{[(2-chloro-4-cyclopropylphenyl)sulfonyl]amino}-4-[(1,1-dimethylethyl)carbamoyl]phenoxy)-2-fluorophenyl]acetic acid.

Other CRTH2 antagonists which may be used in the practice of the invention include those disclosed in U.S. Pat. Appl. Publication No. 2011/0034558. In a particular embodiment, the compound is [2′-(3-benzyl-1-ethyl-ureidomethyl)-6-methoxy-4′-trifluoromethyl-biphenyl-3-yl]-acetic acid and all pharmaceutically acceptable solvates (including hydrates), prodrugs, metabolites, and pharmaceutically acceptable salts thereof.

Other CRTH2 antagonists which may be used in the practice of the invention include those disclosed in International Patent Appl. Publication No. WO 2011/085033. In a particular embodiment, the compound is 2-(3-(2-((tert-butylthio)methyl)-4-(2,2-dimethyl-propionylamino)phenoxy)-4-methoxyphenyl)acetic acid and pharmaceutically acceptable salts, solvates, polymorphs, amorphous phases, and metabolites thereof.

Other CRTH2 antagonists which may be used in the practice of the invention include those disclosed in U.S. Patent Application Publication No. 2010/0173955 having Formula (VIII):

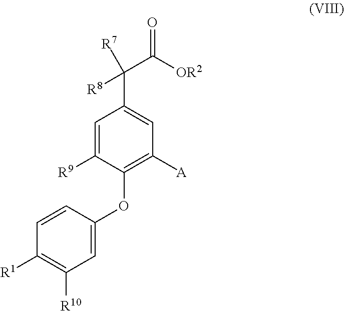

or a salt thereof, wherein: R¹ is Ar¹-L¹-W-L²-; L² is —(CR^{c}R^{d})ₘ—; W is —CONR^{3a}— or —NR^{3b}CO—; R^{3a} and R^{3b} are each H or methyl; L¹ is —(CR^{a}R^{b})ₙ—, —(CH═CH)—, or —O(CR^{a}R^{b}) provided that when W is —NR³CO— then L¹ is not —(CH═CH)—; n and m are independently 0, 1 or 2; each R^{a}, R^{b}, R^{c} and R^{d} is independently H, F, OH, methyl or cyclopropyl, or R^{a} and R^{b} or R^{c} and R^{d} together with the carbon to which they are attached form a cyclopropyl ring; Ar¹ is phenyl or naphthyl, each of which is unsubstituted or substituted with one or more substituents selected independently from F, Cl; CN, CF₃, CHF₂, CH₂F, SF₅, methyl, ethyl, cyclopropyl, t-butyl or OMe, or Ar¹ is 1,2,3,4-tetrahydronaphthyl which is unsubstituted or substituted by methoxy, provided that when Ar¹ is naphthyl or 1,2,3,4-tetrahydronaphthyl then n is 0; R² is H, C₁-C₆ alkyl, a residue of an amino acid or dipeptide, or CHR^{e}(CH₃)_{q}R^{f}; q is 1 to 6; R^{e} is H, methyl or ethyl; R^{f} is NR^{g}R^{h} in which R^{g} and R^{h} each independently represents a hydrogen atom or a C₁-C₄ alkyl group, or R^{g} and R^{h} together with the nitrogen atom to which they are attached form a 5-6 membered heterocyclic ring optionally containing a second ring heteroatom selected from N and O, wherein said heterocyclic ring is optionally substituted with one or more groups independently selected from C₁-C₆ alkyl; A is CN, CH₂NH₂, CH₂NR^{4a}C(═O)R⁵, or CH₂NR^{4b}SO²R⁶, Cl, OMe, (1-4C)alkyl, cyclopropyl, H, F, Br, CH₂NH(1-4C alkyl), CH₂N(1-4C alkyl)₂, thienyl, or phenyl which is unsubstituted or substituted with SO₂Me; R^{4a} and R^{4b} are each H or methyl; R⁵ is C₁-C₆ alkyl, C₁-C₆ alkoxy, C₃-C₆ cycloalkyl, hetAr¹, or Ar²; R⁶ is C₁-C₆ alkyl, NH(C₁-C₆ alkyl), N(C₁-C₆ alkyl)₂, Ar³, or hetAr²; hetAr¹ is a 6 membered heteroaryl which is unsubstituted or substituted with one or more groups independently selected from a halogen atom and a group of formula —NR⁵aR^{5b} in which each of R^{5a} and R^{5b} independently represents a hydrogen atom or a (1-4C) alkyl group, or together with the nitrogen atom to which they are attached form a pyrrolidinyl, piperidinyl or morpholino group; hetAr² is a 5-6 membered heteroaryl which is unsubstituted or substituted with one or more groups independently selected from C₁-C₄ alkyl; Ar² is phenyl which is unsubstituted or substituted with one or more groups independently selected from a halogen atom, CN, SF₅, cyclopropyl, a C₁-C₄ alkyl group, a C₁-C₄ alkoxy group and a fluoroC₁-C₄ alkyl group; Ar³ is as defined for Ar²; R⁷ and R⁸ are independently H, methyl, or F; R⁹ is H or methyl; and R¹⁰ is H or F.

Other CRTH2 antagonists which may be used in the practice of the invention include those disclosed in U.S. Pat. Appl. Publication No. 2011/0034482. In a particular embodiment, the compound is {4,6-bis(dimethyl-amino)-2-(4-(4-(trifluoro-methyl)benzamido)benzyl)pyrimidin-5-yl}acetic acid and pharmaceutically acceptable salts, hydrates, and solvates thereof.

Other CRTH2 antagonists which may be used in the practice of the invention include those disclosed in U.S. Pat. No. 7,696,222 having Formula (IX):

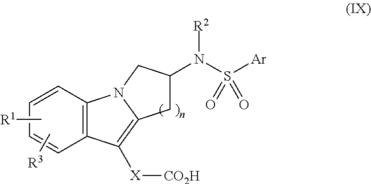

and pharmaceutically acceptable salts thereof, wherein: n is 1 or 2; Ar is aryl or heteroaryl each optionally substituted with 1 to 4 groups independently selected from R^{c}; X is selected from —C(R^{a})(R^{b})—, —C(R^{a})(R^{b})—C(R^{a})(R^{b})—, —C(R^{a})═C(R^{a})—, —OC(R^{a})(R^{b})—, and —SC(R^{a})(R^{b})—; R¹ is selected from H, halogen and C₁₋₆alkyl; R² is selected from H and C₁₋₆alkyl; R³ is selected from H, halogen, C₁₋₆alkyl, OC₁₋₆alkyl, SC₁₋₆alkyl, S(O)ₙC₁₋₆alkyl, CN, aryl and heteroaryl; R^{a} and R^{b} are independently H, halogen, aryl, heteroaryl, C₁₋₆alkyl or haloC₁₋₆alkyl; or R^{a} and R^{b} together with the carbon atom to which they are both attached complete a C₃₋₆cycloalkyl ring; or R^{a} and R^{b} together with the adjacent carbon atoms to which they are attached complete a C₃₋₆cycloalkyl ring; and R^{c} is selected from halogen, CN, C₁₋₆alkoxy, C₁₋₆alkyl, halo C₁₋₆alkoxy, and halo C₁₋₆alkyl. In a particular embodiment, the compound of Formula (IX) is {7-[[4-fluorophenyl)sulfonyl](methyl)amino]-6,7,8,9-tetrahydropyrido[1,2-a]indol-10-yl}acetic acid or a pharmaceutically acceptable salt thereof.

Other CRTH2 antagonists which may be used in the practice of the invention include those disclosed in U.S. Pat. No. 7,858,640 having Formula (X):

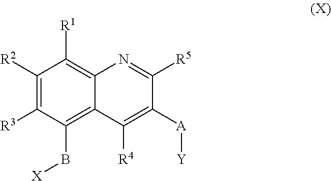

in which: R¹, R², R³, R⁴ and R⁵ are independently hydrogen, C₁-C₆alkyl, fully or partially fluorinated C₁-C₆alkyl, cyclopropyl, halo, —S(OₙR⁶, —SO₂NR⁷R⁸, —NR⁷R⁸, —NR⁷C(O)R⁶, —CO₂R⁷, —C(O)NR⁷R⁸, —C(O)R⁶, —NO₂, —CN or a group —OR⁹; wherein each R⁶ is independently C₁-C₆alkyl, fully or partially fluorinated C₁-C₆alkyl, cycloalkyl, aryl, or heteroaryl; R⁷, R⁵ are independently C₁-C₆alkyl, fully or partially fluorinated C₁-C₆alkyl, cycloalkyl, cycloalkyl-(C₁-C₆alkyl)-, aryl, heteroaryl or hydrogen; R⁹ is hydrogen, C₁-C₆alkyl, fully or partially fluorinated C₁-C₆alkyl, cycloalkyl, cycloalkyl-(C₁-C₆alkyl)-, or a group —SO₂R⁶; A is —CHR¹⁰—, —C(O)—, —S(O)ₙ—, —O—, or —NR¹⁰— wherein n is an integer from 0-2 and R¹⁰ is hydrogen, C₁-C₃alkyl, or fully or partially fluorinated C₁-C₃alkyl group; B is a direct bond, or a divalent radical selected from —CH₂—, —CH₂CH₂—, —CHR¹¹—, —CR¹¹R¹²—, —CH₂CHR¹¹— in either orientation, —CH₂CR¹¹R¹²— in either orientation, —CHR¹¹CHR¹²— in either orientation, and divalent radicals of formula —(CR¹¹R¹²)ₚ—Z— wherein Z is attached to the ring carrying R¹, R² and R³; wherein R¹¹ is C₁-C₃alkyl, cyclopropyl, or fully or partially fluorinated C₁-C₃alkyl; R¹² is methyl or fully or partially fluorinated methyl; p is independently 1 or 2; and Z is —O—, —NH—, or —S(O)ₙ—, wherein n is an integer from 0-2; X is a carboxylic acid, tetrazole, 3-hydroxyisoxazole, hydroxamic acid, phosphinate, phosphonate, phosphonamide, or sulfonic acid group, or a group of formula C(═O)NHSO₂R⁶ or SO₂NHC(═O)R⁶; Y is aryl, heteroaryl, aryl-fused-heterocycloalkyl, heteroaryl-fused-cycloalkyl, heteroaryl-fused-heterocycloalkyl or aryl-fused-cycloalkyl group.

In a particular embodiment, the compound of Formula (X) is selected from the group consisting of a compound selected from the group consisting of: [8-chloro-3-(4-chlorobenzyl)-4-difluoromethoxy-2-ethylquinolin-5-yloxy]acetic acid, [3-(4-chlorobenzyl)-4-difluoromethoxy-2-ethyl-8-fluoroquinolin-5-yloxy]acetic acid, [3-(2,4-dichlorobenzyl)-2-difluoromethoxy-8-fluoro-4-methylquinolin-5-yloxy]acetic acid, [4-difluoromethoxy-2-ethyl-8-fluoro-3-(4-fluorobenzyl)quinolin-5-yloxy]acetic acid, [3-(2,4-difluorobenzyl)-4-difluoromethoxy-2-ethyl-5-fluoroquinolin-5-yloxy]acetic acid, [3-(2,4-dichlorobenzyl)-4-difluoromethoxy-2-ethyl-8-fluoroquinolin-5-yloxy]acetic acid, [3-(4-chloro-2-fluorobenzyl)-2-difluoromethoxy-8-fluoro-4-methylquinolin-5-yloxy]acetic acid, [8-chloro-3-(4-chlorobenzyl)-2-difluoromethoxy-4-methylquinolin-5-yloxy]acetic acid, [3-(2-chloro-4-fluorobenzyl)-4-difluoromethoxy-2-ethyl-8-fluoroquinolin-5-yloxy]acetic acid, [3-(2-chloro-4-fluorobenzyl)-2-difluoromethoxy-8-fluoro-4-methylquinolin-5-yloxy]acetic acid, [8-chloro-3-(4-chloro-2-fluorobenzyl)-4-difluoromethoxy-2-ethylquinolin-5-yloxy]acetic acid, [3-(4-chloro-2-fluorobenzyl)-4-difluoromethoxy-2-ethyl-8-fluoroquinolin-5-yloxy]acetic acid, {4-difluoromethoxy-2-ethyl-8-fluoro-3-[4-(morpholine-4-sulfonyl)benzyl]quinolin-5-yloxy}acetic acid, {4-difluoromethoxy-2-ethyl-8-fluoro-3-[4-(pyrrolidine-1-carbonyl)benzyl]quinolin-5-yloxy}acetic acid, 2-[3-(2,4-dichlorobenzyl)-2-difluoromethoxy-8-fluoro-4-methylquinolin-5-yloxy]propionic acid, (S)-2-[3-(2,4-dichlorobenzyl)-2-difluoromethoxy-8-fluoro-4-methylquinolin-5-yloxy]propionic acid, 2-[8-chloro-3-(4-chlorobenzyl)-2-difluoromethoxy-4-methylquinolin-5-yloxy]propionic acid, {8-chloro-4-difluoromethoxy-2-ethyl-3-[4-(pyrrolidine-1-carbonyl)benzyl]-quinolin-5-yloxy}acetic acid, {3-[2-chloro-4-(pyrrolidine-1-carbonyl)benzyl]-4-difluoromethoxy-2-ethyl-8-fluoroquinolin-5-yloxy}acetic acid, (S)-2-{3-[2-chloro-4-(pyrrolidine-1-carbonyl)benzyl]-4-difluoromethoxy-2-ethyl-8-fluoroquinolin-5-yloxy}propionic acid, (S)-2-[3-(2,4-dichlorobenzyl)-4-difluoromethoxy-2-ethyl-8-fluoroquinolin-5-yloxy]propionic acid, [3-(2-chloro-4-cyclobutylcarbamoylbenzyl)-4-difluoromethoxy-2-ethyl-8-fluoroquinolin-5-yloxy]acetic acid; and (S)-2-[3-(2-chloro-4-cyclobutylcarbamoylbenzyl)-4-difluoromethoxy-2-ethyl-8-fluoroquinolin-5-yloxy]propionic acid; and pharmaceutically acceptable salts and N-oxides thereof.

See also the following published applications which disclose CRTH2 antagonists: WO-A-03/066046, WO-A-03/066047, WO-A-03/097042, WO-A-03/097598, WO-A-03/101981, WO-A-03/101961, WO-A-2004/007451, WO-A-2005/019171, WO-A-2005/054232, WO-A-2004/089884, WO-A-2004/089885, WO-A-2005/018529, WO-A-2006/005909, WO2006/021759, WO-A-2007/039736, WO-A-2007/052023, WO-A-2006/075139, WO-A-2007/068894, WO-A-2007138282, WO-A-2008/119917, WO-A-2008/113965, WO-A-2008/074966, WO-A-2008/078069, WO-A-2007/144625, WO-A-2007/028999, WO-A-2007/031747, WO-A-2006/136859, WO-A-2006/111560, WO-A-2005/094816, WO-A-2005/040112, WO-A-2005/040114, WO-A-2004/096777, WO-A-2005/123731, WO-A-2006/125784, WO-A-2007/045867, WO-A-2006/034419, WO-A-2006/036994, WO-A-2007/022501, WO-A-2004/106302, WO-A-2004/032848, WO-A-2005/100321, WO-A-2006/091674, WO-A-2004/058164, WO-A-2005/007094, WO-A-2007/036743, WO-2004/035543, WO-A-2007/062797, WO-A-2007/062773, WO-A-2007/062678, WO-A-2007/062677, WO-A-2005/116001, WO-A-2005/115382, WO-A-2005/115374, WO-A-2006/11560, WO-A-2006/037982, WO-A-2006/056752, WO-A-2007/039741, WO-A-2005/073234, WO-A-2005/105727, WO-A-2006/063763, WO-A-2006/125593 and WO-A-2006/125596.

In one embodiment, the proton pump inhibitor (PPI) is disclosed in U.S. Pat. No. 4,045,563 and has Formula (XI)

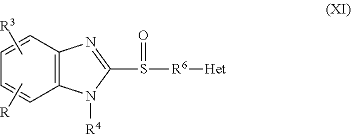

wherein R and R³ are the same or different and are selected from the group consisting of hydrogen, alkyl, halogen, cyano, carboxy, carboxy-alkyl, carboalkoxy, carbo-alkoxyalkyl, carbamoyl, carbamoyloxy, hydroxy, alkoxy, hydroxy alkyl, trifluoromethyl and acyl in any position, R⁴ is selected from the group consisting of hydrogen, alkyl, acyl, carboalkoxy, carbamoyl, alkylcarbamoyl, dialkylcarbamoyl, alkylcarbonyl methyl, alkoxycarbonyl methyl and alkylsulphonyl, R⁶ is selected from the group consisting of a straight or branched alkyl chain having 1 to 4 carbon atoms, whereby only one methylene group is present between S and Het, and Het is selected from the group consisting of imidazolyl, imidazolinyl, benzimidazolyl, thiazolyl, thiazolinyl, quinolyl, piperidyl and pyridyl, which may be further substituted preferably in the 3 to 5 position with lower alkyl groups such as methyl, ethyl and propyl and/or with halo substituents such as chloro and bromo, and pharmaceutically acceptable salts.

Examples of compounds having Formula (XI) include 2-[2-pyridylmethylsulfinyl]-benzimidazole, 2-[2-pyridylmethylsulfinyl]-(4,6-dimethyl)benzimidazole, 2-[2-pyridylmethylsulfinyl]-(5-ethyl)benzimidazole, 2-[2-pyridylmethylsulfinyl]-(4-methyl, 6-chloro)benzimidazole, 2-[2-pyridylmethylsulfinyl]-(5-methoxy)benzimidazole, 2-[2-pyridylmethylsulfinyl]-(5-hydroxy)benzimidazole, 2-[2-pyridylmethylsulfinyl]-(5-acetyl)benzimidazole, 2-[2-pyridylmethylsulfinyl]-(5-carboxy)benzimidazole, 2-[2-pyridylmethylsulfinyl]-(5-carbethoxy)benzimidazole, 2-[2-(4-chloro)pyridylmethylsulfinyl]benzimidazole, 2-[2-(5-methyl)pyridylmethylsulfinyl]benzimidazole, 2-[2-pyridylmethylsulfinyl]-N-methylbenzimidazole, 2-[2-pyridyl-(methyl)methylsulfinyl]benzimidazole, 2-[2-pyridylmethylsulfinyl]-(4-methyl)benzimidazole, 2-[2-pyridylmethylsulfinyl]-(N-acetyl)benzimidazole, 2-[2-pyridylmethylsulfinyl]-(N-methoxycarbonyl)benzimidazole, 2-[2-pyridylmethylsulfinyl]-(5-methyl)benximidazole, 2-[2-pyridylmethylsulfinyl]-(5-chloro)benzimidazole, 2-[2-pyridylmethylsulfinyl]-(5-isopropyl)benzimidazole, 2-[2-pyridylmethylsulfinyl]-(5-t-butyl)benzimidazole, 2-[2-pyridylmethylsulfinyl]-(5-n-propyl)benzimidazole, 2-[2-pyridylmethylsulfinyl]-(N-carbamoyl)benzimidazole, 2-[2-pyridylmethylsulfinyl]-(N-methylcarbamoyl)benzimidazole, 2-[2-pyridylmethylsulfinyl]-(N-acetylmethyl)benzimidazole, 2-[2-pyridylmethylsulfinyl]-(N-ethoxycarbonylmethyl)benzimidazole, 2-[2-pyridylmethylsulfinyl]-(N-methylsulfonyl)benzimidazole, 2-[2-(4-methyl)pyridylmethylsulfinyl]-(5-methyl)benzimidazole, 2-[2-(5-methyl)pyridylmethylsulfinyl]-(5-methyl)benzimidazole, 2-[2-pyridylmethylsulfinyl]-(6-chloro)benzimidazole, 2-[2-pyridyl-(ethyl)methylsulfinyl]-benzimidazol, 2-[2-pyridyl-(ethyl)methylsulfinyl]-(5-chloro)benzimidazole, 2-[2-pyridyl-(methyl)methylsulfinyl]-(5-ethyl)benzimidazole, 2-[2-(3-methyl)pyridylmethylsulfinyl]benzimidazole, 2-[2-(5-ethyl)pyridylmethylsulfinyl]-(5-methyl)benzimidazole, 2-[2-5 ethyl)pyridylmethylsulfinyl]benzimidazole, 2-[2-pyridyl-(ethyl)methylsulfinyl]-(5-ethyl)benzimidazole, 2-[2-pyridyl-(methyl)methylsulfinyl]-(5-methyl)benzimidazole, 2-[2-pyridyl-(methyl)methylsulfinyl]-(5-cyano)benzimidazole, 2-[2-pyridyl-(methyl)methylsulfinyl]-(5-trifluoro)benzimidazole, 2-[2-pyridyl-(ethyl)methylsulfinyl]-(5-methyl)benzimidazole, 2-[2-pyridyl-(ethyl)methylsulfinyl]-(5-cyano)benzimidazole, 2-[2-pyridyl-(ethyl)methylsulfinyl]-(5-trifluoro)benzimidazole, 2-[2-pyridylmethylsulfinyl]-(4-chloro)benzimidazole, 2-[2-pyridyl-(isopropyl)methylsulfinyl]benzimidazole, 2-[2-pyridyl-(methyl)methylsulfinyl]-(5,6-dimethyl)benzimidazole, and 2-[2-pyridylmethylsulfinyl]-(5,6-dimethyl)benzimidazole.

In another embodiment, the PPI is disclosed in U.S. Pat. No. 4,853,230 and has Formula (XII):

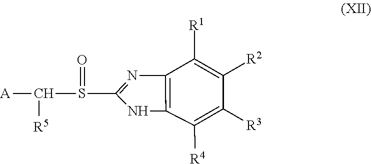

wherein A is an optionally substituted heterocyclic group, R¹, R², R³ and R⁴ are the same or different and select from among hydrogen, lower alkyl, lower alkoxy, —CF₃,

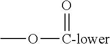

alkyl or halogen and R⁵ is H or a lower alkyl group wherein “lower” denotes 1-6 carbon atoms, and pharmaceutically acceptable salts thereof.

Examples of compounds of Formula (XII) include (RS)-6-methoxy-2-((4-methoxy-3,5-dimethylpyridin-2-yl)methylsulfinyl)-1H-benzo[d]imidazole.

In another embodiment, the PPI is the (S)-enantiomer of 5-methoxy-2-[[(4-methoxy-3,5-dimethylpyridin-2-yl)methyl]sulfinyl]-1H-benzo[d]imidazole or the alkaline salt thereof as disclosed in U.S. Pat. No. 5,714,504.

In another embodiment, the PPI is disclosed in U.S. Pat. No. 4,628,098 and has Formula XIII:

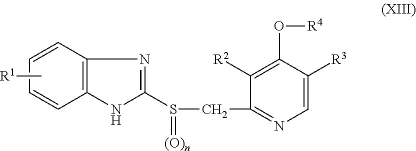

and the pharmaceutically acceptable salts thereof, wherein R¹ is hydrogen, methoxy, or trifluoromethyl, R² and R³ are independently hydrogen or methyl, R⁴ is a C₂₋₅ fluorinated alkyl, and n denotes 0 or 1, and the pharmaceutically acceptable salts thereof.

Examples of compounds of Formula XIII include 2-[4-(2,2,2-trifluoroethoxy)-pyrid-2-yl]methylsulfinylbenzimidazole, 2-[3-methyl-4-(2,2,2-trifluoroethoxy)-pyrid-2-yl]methylsulfinylbenzimidazole, 2-[4-(2,2,2-trifluoroethoxy)-5-methyl-pyrid-2-yl]methylsulfinylbenzimidazole, 2-[3-methyl-4-(2,2,2-trifluoroethoxy)-5-methyl-pyrid-2-yl]methylsulfinylbenzimidazole, 2-[4-(2,2,3,3,3-pentafluoropropoxy)-pyrid-2-yl]methylsulfinylbenzimidazole, 2-[4-(2,2,3,3,3-pentafluoropropoxy)-5-methyl-pyrid-2-yl]methylsulfinylbenzimidazole, 2-[4-(2,2,3,3-tetrafluoropropoxy)-pyrid-2-yl]methylsulfinylbenzimidazole, 2-[3-methyl-4-(2,2,3,3,3-pentafluoropropoxy)-pyrid-2-yl]methylsulfinylbenzimidazole, 2-[3-methyl-4-(2,2,3,3-tetrafluoropropoxy)-pyrid-2-yl]methylsulfinylbenzimidazole, 2-[5-methyl-4-(2,2,3,3-tetrafluoropropoxy)-pyrid-2-yl]methylsulfinylbenzimidazole, 2-[3,5-dimethyl-4-(2,2,3,3,3-pentafluoropropoxy)-pyrid-2-yl]methylsulfinylbenzimidazole, 2-[3-methyl-4-(2,2,2-trifluoroethoxy)-pyrid-2-yl]methylsulfinyl-5-trifluoromethylbenzimidazole, 2-[3-methyl-4-(2,2,2-trifluoroethoxy)-pyrid-2-yl]methylsulfinyl-5-methoxybenzimidazole, 2-[4-(2,2,2-trifluoroethoxy)-pyrid-2-yl]-methylsulfinyl-5-methoxybenzimidazole, 2-[4-(2,2,2-trifluoroethoxy)-pyrid-2-yl]-methylthiobenzimidazole, 2-[3-methyl-4-(2,2,2-trifluoroethoxy)-pyrid-2-yl]methylthiobenzimidazole, 2-[5-methyl-4-(2,2,2-trifluoroethoxy)-pyrid-2-yl]methylthiobenzimidazole, 2-[3,5-dimethyl-4(2,2,2-trifluoroethoxy)-pyrid-2-yl]methylthiobenzimidazole, 2-[4-(2,2,3,3,3-pentafluoropropoxy)-pyrid-2-yl]methylthiobenzimidazole, 2-[5-methyl-4-(2,2,3,3,3-pentafluoropropoxy)-pyrid-2-yl]methylthiobenzimidazole, 2-[4-(2,2,3,3-tetrafluoropropoxy)-pyrid-2-yl]methylthiobenzimidazole, 2-[3-methyl-4-(2,2,3,3-tetrafluoropropoxy)-pyrid-2-yl]methylthiobenzimidazole, 2-[5-methyl-4-(2,2,3,3-tetrafluoropropoxy)-pyrid-2-yl]methylthiobenzimidazole, 2-[3-methyl-4-(2,2,3,3,3-pentafluoropropoxy)-pyrid-2-yl]methylthiobenzimidazole, 2-[3-methyl-4-(2,2,3,3,3-pentafluoropropoxy)-5-methyl-pyrid-2-yl]methylthiobenzimidazole, 2-[3-methyl-4-(2,2,2-trifluoroethoxy)-pyrid-2-yl-methylthio-5-trifluoromethylbenzimidazole, 2-[3-methyl-4-(2,2,2-trifluoroethoxy)-pyrid-2-yl-methylthio-5-methoxybenzimidazole, and 2-[4-(2,2,2-trifluoroethoxy)-pyrid-2-yl]methylthio-5-methoxybenzimidazole, and the pharmaceutically acceptable salts thereof.

In another embodiment, the PPI is disclosed in U.S. Pat. No. 4,758,579 and has Formula (XIV):

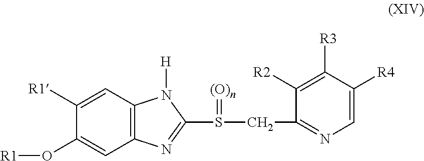

and the pharmaceutically acceptable salts thereof, wherein R1 represents a 1-3C-alkyl radical which is completely or predominantly substituted by fluorine, or a chlorodifluoromethyl radical and R1′ represents hydrogen (—H), halo, trifluoromethyl, a 1-3C-alkyl radical, or a 1-3C-alkoxy radical which is, optionally, completely or predominantly substituted by fluorine, or R1 and R1′ together, with inclusion of the oxygen atom to which R1 is bonded, represent a 1-2C-alkylenedioxy radical which is, optionally, completely or partly substituted by fluorine, or a chlorotrifluoroethylenedioxy radical, R3 represents a 1-3C-alkoxy radical, one of the radicals R2 and R4 represents a 1-3C-alkoxy radical and the other represents a hydrogen atom (H) or a 1-3C-alkyl radical and n represents the number 0 or 1.

Examples of compounds of Formula (XIV) include 2-[(4,5-dimethoxy-3-methyl-2-pyridyl)methylsulfinyl]-5-trifluoromethoxy-1H-benzimidazole, 2-[(4,5-dimethoxy-3-methyl-2-pyridyl)-methylthio]-5-trifluoromethoxy-1H-benzimidazole, 2-[(4,5-dimethoxy-3-methyl-2-pyridyl)methylsulfinyl]-5-(1,1,2,2-tetrafluoroethoxy)-1H-benzimidazole, 2-[(4,5-dimethoxy-3-methyl-2-pyridyl)methylthio]-5-(1,1,2,2-tetrafluoroethoxy)-1H-benzimidazole, 2-[(4,5-dimethoxy-3-methyl-2-pyridyl)methylsulfinyl]-5-(2,2,2-trifluoroethoxy)-1H-benzimidazole, 2-[(4,5-dimethoxy-3-methyl-2-pyridyl)-methylthio]-5-(2,2,2-trifluoroethoxy)-1H-benzimidazole, 5-difluoromethoxy-2-[(4,5-dimethoxy-3-methyl-2-pyridyl)methylsulfinyl]-1H-benzimidazole, 5-difluoromethoxy-2-[(4,5-dimethoxy-3-methyl-2-pyridyl)methylthio]-1H-benzimidazole, 5-chlorodifluoromethoxy-2-[(4,5-dimethoxy-3-methyl-2-pyridyl)methylsulfinyl]-1H-benzimidazole, 5-chlorodifluoromethoxy-2-[(4,5-dimethoxy-3-methyl-2-pyridyl)methylthio]-1H-benzimidazole, 5,6-bis(difluoromethoxy)-2-[(4,5-dimethoxy-3-methyl-2-pyridyl)methylsulfinyl]-1H-benzimidazole, 5,6-bis(difluoromethoxy)-2-[(4,5-dimethoxy-3-methyl-2-pyridyl)methylthio]-1-H-benzimidazole, 5-difluoromethoxy-6-methoxy-2-[(4,5-dimethoxy-3-methyl-2-pyridyl)methylsulfinyl]-1H-benzimidazole, 5-difluoromethoxy-6-methoxy-2-[(4,5-dimethoxy-3-methyl-2-pyridyl)-methylthio]-1H-benzimidazole, 2-[(4,5-dimethoxy-2-pyridyl)methylsulfinyl]-5-trifluoromethoxy-1H-benzimidazole, 2-[(4,5-dimethoxy-2-pyridyl)methylthio]-5-trifluoromethoxy-1H-benzimidazole 2-[(4,5-dimethoxy-2-pyridyl)methylsulfinyl]-5-(1,1,2,2-tetrafluoroethoxy)-1H-benzimidazole, 2-[(4,5-dimethoxy-2-pyridyl)methylthio]-5-(1,1,2,2-tetrafluoroethoxy)-1H-benzimidazole, 2-[(4,5-dimethoxy-2-pyridyl)methylsulfinyl]-5-(2,2,2-trifluoroethoxy)-1H-benzimidazole, 2-[(4,5-dimethoxy-2-pyridyl)methylthio]-5-(2,2,2-trifluoroethoxy)-1H-benzimidazole, 5-difluoromethoxy-2-[(4,5-dimethoxy-2-pyridyl)methylsulfinyl]-1H-benzimidazole, 5-difluoromethoxy-2-[(4,5-dimethoxy-2-pyridyl)methylthio]-1H-benzimidazole, 5-chlorodifluoromethoxy-2-[(4,5-dimethoxy-2-pyridyl)methylsulfinyl]-1H-benzimidazole, 5-chlorodifluoromethoxy-2-[(4,5-dimethoxy-2-pyridyl)methylthio]-1H-benzimidazole, 5,6-bis(difluoromethoxy)-2-[(4,5-dimethoxy-2-pyridyl)methylsulfinyl]-1H-benzimidazole, 5,6-bis(difluoromethoxy)-2-[(4,5-dimethoxy-2-pyridyl)methylthio]-1H-benzimidazole, 5-difluoromethoxy-6-methoxy-2-[(4,5-dimethoxy-2-pyridyl)methylsulfinyl]-1H-benzimidazole, 5-difluoromethoxy-6-methoxy-2-[4,5-dimethoxy-2-pyridyl)methylthio]-1H-benzimidazole, 2-[(3,4-dimethoxy-5-methyl-2-pyridyl)methylsulfinyl]-5-trifluoromethoxy-1H-benzimidazole, 2-[(3,4-dimethoxy-5-methyl-2-pyridyl)methylthio]-5-trifluoromethoxy-1H-benzimidazole, 2-[(3,4-dimethoxy-5-methyl-2-pyridyl)methylsulfinyl]-5-(1,1,2,2-tetrafluoroethoxy)-1H-benzimidazole, 2-[(3,4-dimethoxy-5-methyl-2-pyridyl)methylthio]-5-(1,1,2,2-tetrafluoro-ethoxy)-1H-benzimidazole, 2-[(3,4-dimethoxy-5-methyl-2-pyridyl)methylsulfinyl]-5-(2,2,2-trifluoroethoxy)-1H-benzimidazole, 2-[(3,4-dimethoxy-5-methyl-2-pyridyl)-methylthio]-5-(2,2,2-trifluoroethoxy)-1H-benzimidazole, 5-difluoromethoxy-2-[(3,4-dimethoxy-5-methyl-2-pyridyl)methylsulfinyl]-1H-benzimidazole, 5-difluoromethoxy-2-[(3,4-dimethoxy-5-methyl-2-pyridyl)methylthio]-1H-benzimidazole, 5-chlorodifluoromethoxy-2-[(3,4-dimethoxy-5-methyl-2-pyridyl)methylsulfinyl]-1H-benzimidazole, 5-chlorodifluoromethoxy-2-[(3,4-dimethoxy-5-methyl-2-pyridyl)methylthio]-1H-benzimidazole, 5,6-bis(difluoromethoxy)-2-[(3,4-dimethoxy-5-methyl-2-pyridyl)methylsulfinyl]-1H-benzimidazole, 5,6-bis(difluoromethoxy)-2-[(3,4-dimethoxy-5-methyl-2-pyridyl)methylthio]-1H-benzimidazole, 5-difluoromethoxy-6-methoxy-2-[((3,4-dimethoxy-5-methyl-2-pyridyl)methylsulfinyl]-1H-benzimidazole, 5-difluoromethoxy-6-methoxy-2-[(3,4-dimethoxy-5-methyl-2-pyridyl)methylthio]-1H-benzimidazole, 2-[(3,4-dimethoxy-2-pyridyl)methylsulfinyl]-5-trifluoromethoxy-1H-benzimidazole, 2-[(3,4-dimethoxy-2-pyridyl)methylthio]-5-trifluoromethoxy-1H-benzimidazole, 2-[(3,4-dimethoxy-2-pyridyl)methylsulfinyl]-5-(1,1,2,2-tetrafluoroethoxy)-1H-benzimidazole, 2-[(3,4-dimethoxy-2-pyridyl)methylthio]-5-(1,1,2,2-tetrafluoroethoxy)-1H-benzimidazole, 2-[(3,4-dimethoxy-2-pyridyl)methylsulfinyl]-5-(2,2,2-trifluoroethoxy)-1H-benzimidazole, 2-[(3,4-dimethoxy-2 pyridyl)methylthio]-5-(2,2,2-trifluoroethoxy)-1H-benzimidazole, 5-difluoromethoxy-2-[(3,4-dimethoxy-2-pyridyl)methylsulfinyl]-1H-benzimidazole, 5-difluoromethoxy-2-[(3,4-dimethoxy-2-pyridyl)methylthio]-1H-benzimidazole, 5-chlorodifluoromethoxy-2-[(3,4-dimethoxy-2-pyridyl)methylsulfinyl]-1H-benzimidazole, 5-chlorodifluoromethoxy-2-[(3,4-dimethoxy-2-pyridyl)methylthio]-1H-benzimidazole, 5,6-bis(difluoromethoxy)-2-[(3,4-dimethoxy-2-pyridyl)methylsulfinyl]-1H-benzimidazole, 5,6-bis(difluoromethoxy)-2-[(3,4-dimethoxy-2-pyridyl)methylthio]-1H-benzimidazole, 5-difluoromethoxy-6-methoxy-2-[(3,4-dimethoxy-2-pyridyl)methylsulfinyl]-1H-benzimidazole, 5-difluoromethoxy-6-methoxy-2-[(3,4-dimethoxy-2-pyridyl)methylthio]-1H-benzimidazole, 2,2-difluoro-6-[(4,5-dimethoxy-2-pyridyl)methylsulfinyl]-5H-[1,3]-dioxolo-[4,5-f]benzimidazole, 2,2-difluoro-6-[(4,5-dimethoxy-2-pyridyl)methylthio]-5H-[1,3]-dioxolo-[4,5-f]benzimidazole, 2,2-difluoro-6-[(3-methyl-4,5-dimethoxy-2-pyridyl)methylthio]-5H-[1,3]-dioxolo[4,5-f]benzimidazole, 2,2-difluoro-6-[(3-methyl-4,5-dimethoxy-2-pyridyl)methylsulfinyl]-5H-[1,3]-dioxolo[4,5-f]benzimidazole, 6-[(4,5-diethoxy-3-methyl-2-pyridyl)methylthio]-2,2-difluoro-5H-[1,3]-dioxolo[4,5-f]benzimidazole, 6-[(4,5-diethoxy-3-methyl-2-pyridyl)methylsulfinyl]-2,2-difluoro-5H-[1,3]-dioxolo[4,5-f]benzimidazole, 6,6,7-trifluoro-6,7-dihydro-2-[(4,5-dimethoxy-3-methyl-2-pyridyl)methylthio-1H-[1,4]-dioxino[2,3-f]benzimidazole, 6,6,7-trifluoro-6,7-dihydro-2-[(4,5-dimethoxy-3-methyl-2-pyridyl)methylsulfinyl]-1H-[1,4]-dioxino[2,3-f]benzimidazole, 6,6,7-trifluoro-6,7-dihydro-2-[(4,5-dimethoxy-2-pyridyl)methylthio]-1H-[1,4]-dioxino-[2,3-f]benzimidazole, 6,6,7-trifluoro-6,7-dihydro-2-[(4,5-dimethoxy-2-pyridyl)methylsulfinyl]-1H-[1,4]-dioxino[2,3-f]benzimidazole, 2-[(4,5-diethoxy-2-pyridyl)methylthio]6,6,7-trifluoro-6,7-dihydro-1H-[1,4]-dioxino[2,3-f]benzimidazole, 2-[(4,5-diethoxy-2-pyridyl)methylsulfinyl]-6,6,7-trifluoro-6,7-dihydro-1H-[1,4]-dioxino[2,3-f]benzimidazole, 2-[(4,5-diethoxy-3-methyl-2-pyridyl)methylthio]-6,6,7-trifluoro-6,7-dihydro-1H-[1,4]-dioxino[2,3-f]benzimidazole, 2-[(4,5-diethoxy-3-methyl-2-pyridyl)methylsulfinyl]-6,6,7-trifluoro-6,7-dihydro-1H-[1,4]-dioxino[2,3-f]benzimidazole, 6,6-difluoro-6,7-dihydro-2-[(4,5-dimethoxy-2-pyridyl)methylthio]-1H-[1,4]-dioxino[2,3-f]benzimidazole, 6,6-difluoro-6,7-dihydro-2-[(4,5-dimethoxy-2-pyridyl)methylsulfinyl]-1H-[1,4]-dioxinio[2,3-f]benzimidazole, 6,6-difluoro-6,7-dihydro-2-[(4,5-dimethoxy-3-methyl-2-pyridyl)methylthio]-1H-[1,4]-dioxino[2,3-f]benzimidazole, 6,6-difluoro-6,7-dihydro-2-[(4,5-dimethoxy-3-methyl-2-pyridyl)methylsulfinyl]-1H-[1,4]-dioxino[2,3-f]benzimidazole, 6,6,7,7-tetrafluoro-6,7-dihydro-2-[(4,5-dimethoxy-2-pyridyl)methylthio]-1H-[1,4]-dioxino[2,3-f]benzimidazole, 6,6,7,7-tetrafluoro-6,7-dihydro-2-[(4,5-dimethoxy-2-pyridyl)methylsulfinyl]-1H-[1,4]-dioxino[2,3-f]benzimidazole, 6,6,7,7-tetrafluoro-6,7-dihydro-2-[(4,5-dimethoxy-3-methyl-2-pyridyl)methylthio]-1H-[1,4]-dioxino[2,3-t]benzimidazole, 6,6,7,7-tetrafluoro-6,7-dihydro-2-[(4,5-dimethoxy-3-methyl-2-pyridyl)methylsulfinyl]-1H-[1,4]-dioxine[2,3-f]benzimidazole, 6-chloro-6,7,7-trifluoro-6,7-dihydro-2-[(4,5-dimethoxy-3-methyl-2-pyridyl)methylsulfinyl]-1H-[1,4]dioxino[2,3-f]benzimidazole, 6-chloro-6,7,7-trifluoro-6,7-dihydro-2-[(4,5-dimethoxy-3-methyl-2-pyridyl)methylthio]-1H-[1,4]-dioxino[2,3-t]benzimidazole, 6-chloro-6,7,7-trifluoro-6,7-dihydro-2-[(4,5-dimethoxy-2-pyridyl)methylsulfinyl]-1H-[1,4]-dioxino[2,3-f]-benzimidazole, 6-chloro-6,7,7-trifluoro-6,7-dihydro-2-[(4,5-dimethoxy-2-pyridyl)methylthio]-1H-[1,4]-dioxino[2,3-f]benzimidazole, 2,2-difluoro-6-[(3,4-dimethoxy-2-pyridyl)methylsulfinyl]-5H-[1,3]-dioxolo[4,5-f]benzimidazole, 2,2-difluoro-6-[(3,4-dimethoxy-2-pyridyl)methylthio]-5H-[1,3]-dioxolo-[4,5-f]benzimidazole, 2,2-difluoro-6-[(3,4-dimethoxy-5-methyl-2-pyridyl)methylthio]-5H-[1,3]-dioxolo[4,5-f]benzimidazole, 2,2-difluoro-6-[(3,4-dimethoxy-5-methyl-2-pyridyl)methylsulfinyl]-5H-[1,3]-dioxolo[4,5-f]benzimidazole, 6-[(3,4-diethoxy-5-methyl-2-pyridyl)methylthio]-2,2-difluoro-5H-[1,3]-dioxolo[4,5-f]benzimidazole, 6-[(3,4-diethoxy-5-methyl-2-pyridyl)methylsulfinyl]-2,2-difluoro-5H-[1,3]-dioxolo[4,5-f]benzimidazole, 6,6,7-trifluoro-6,7-dihydro-2-[(3,4-dimethoxy-5-methyl-2-pyridyl)methylthio]-1H-[1,4]-dioxino[2,3-t]benzimidazole, 6,6,7-trifluoro-6,7-dihydro-2-[(3,4-dimethoxy-5-methyl-2-pyridyl)methylsulfinyl]-1H-[1,4]-dioxino[2,3-t]benzimidazole, 6,6,7-trifluoro-6,7-dihydro-2-[(3,4-dimethoxy-2-pyridyl)methylthio]-1H-[1,4]-dioxino[2,3-f]benzimidazole, 6,6,7-trifluoro-6,7-dihydro-2-[(3,4-dimethoxy-2-pyridyl)methylsulfinyl]-1H-[1,4]-dioxino[2,3-f]benzimidazole, 2-[(3,4-diethoxy-2-pyridyl)methylthio]-6,6,7-trifluoro-6,7-dihydro-1H-[1,4]-dioxino[2,3-f]benzimidazole, 2-[(3,4-diethoxy-2-pyridyl)methylsulfinyl]-6,6,7-trifluoro-6,7-dihydro-1H-[1,4]-dioxino[2,3-f]benzimidazole, 2-[(3,4-diethoxy-5-methyl-2-pyridyl)methylthio]-6,6,7-trifluoro-6,7-dihydro-1H-[1,4]-dioxino[2,3-f]benzimidazole, 2-[(3,4-diethoxy-5-methyl-2-pyridyl)methylsulfinyl]-6,6,7-trifluoro-6,7-dihydro-1H-[1,4]-dioxino[2,3-f]benzimidazole, 6,6-difluoro-6,7-dihydro-2-[(3,4-dimethoxy-2-pyridyl)methylthio]-1H-[1,4]-dioxino[2,3-f]benzimidazole, 6,6-difluoro-6,7-dihydro-2-[(3,4-dimethoxy-2-pyridyl)methyl-sulfinyl]-1H-[1,4]-dioxino[2,3-f]benzimidazole, 6,6-difluoro-6,7-dihydro-2-[(3,4-dimethoxy-5-methyl-2-pyridyl)methylthio]-1H-[1,4]-dioxino[2,3-f]benzimidazole, 6,6-difluoro-6,7-dihydro-2-[(3,4-dimethoxy-5-methyl-2-pyridyl)methylsulfinyl]-1H-[1,4]-dioxino[2,3-f]benzimidazole, 6,6,7,7-tetrafluoro-6,7-dihydro-2-[(3,4-dimethoxy-2-pyridyl)methylthio]-1H-[1,4]-dioxino[2,3-f]benzimidazole, 6,6,7,7-tetrafluoro-6,7-dihydro-2-[(3,4-dimethoxy-2-pyridyl)methylsulfinyl]-1H-[1,4]-dioxinio[2,3-f]benzimidazole, 6,6,7,7-tetrafluoro-6,7-dihydro-2-[(3,4-dimethoxy-5-methyl-2-pyridyl)methylthio]-1H-[1,4]-dioxino[2,3-f]benzimidazole, 6,6,7,7-tetrafluoro-6,7-dihydro-2-[(3,4-dimethoxy-5-methyl-2-pyridyl)methylsulfinyl]-1H-[1,4]-dioxino[2,3-f]benzimidazole, 6-chloro-6,7,7-trifluoro-6,7-dihydro-2-[(3,4-dimethoxy-5-methyl-2-pyridyl)methylsulfinyl]-1H-[1,4]-dioxino[2,3-f]benimidazole, 6-chloro-6,7,7-trifluoro-6,7-dihydro-2-[(3,4-dimethoxy-5-methyl-2-pyridyl)methylthio]-1H-[1,4]-dioxino[2,3-f]benzimidazole, 6-chloro-6,7,7-trifluoro-6,7-dihydro-2-[(3,4-dimethoxy-2-pyridyl)methylsulfinyl]-1H-[1,4]-dioxino[2,3-f]benzimidazole, 6-chloro-6,7,7-trifluoro-6,7-dihydro-2-[(3,4-dimethoxy-2-pyridyl)methylthio]-1H-[1,4]-dioxino[2,3-f]benzimidazole, 6-[(4,5-dimethoxy-3-methyl-2-pyridyl)-methylthio]-5H-[1,3]dioxolo[4,5-f]benzimidazole, 6-[(4,5-dimethoxy-3-methyl-2-pyridyl)-methylsulfinyl]-5H-[1,3]-dioxolo[4,5-f]benzimidazole, 6-[(4,5-dimethoxy-2-pyridyl)methylthio]-5H-[1,3]dioxolo[4,5-d]benzimidazole 6-[(4,5-dimethoxy-2-pyridyl)methyl-sulfinyl]-5H-[1,3]-dioxolo[4,5-f]benzimidazole, 6-[(3,4-dimethoxy-2-pyridyl)-methylthio]-5H-[1,3]-dioxolo[4,5-f]benzimidazole, 6-[(3,4-dimethoxy-2-pyridyl)methylsulfinyl]-5H-[1,3]-dioxolo[4,5-f]benzimidazole, 6-[(3,4-dimethoxy-5-methyl-2-pyridyl)methylthio]-5H-[1,3]-dioxolo[4,5-f]-benzimidazole, 6-[(3,4-dimethoxy-5-methyl-2-pyridyl)methylsulfinyl]-5H-[1,3]-dioxolo[4,5-f]benzimidazole, 6,7-dihydro-2-[(4,5-dimethoxy-3-methyl-2-pyridyl)methylthio]-1H-[1,4]-dioxino[2,3-f]benzimidazole, 6,7-dihydro-2-[(4,5-dimethoxy-3-methyl-2-pyridyl)methylsulfinyl]-1H-[1,4]-dioxino[2,3-f]benzimidazole, 6,7-dihydro-2-[(3,4-dimethoxy-5-methyl-2-pyridyl)methylthio]-1H-[1,4]-dioxino[2,3-f]benzimidazole, 6,7-dihydro-2-[(3,4-dimethoxy-5-methyl-2-pyridyl)methylsulfinyl]-1H-[1,4]-dioxino[2,3-f]benzimidazole, 6,7-dihydro-2-[(3,4-dimethoxy-2-pyridyl)methylthio]-1H-[1,4]-dioxino[2,3-f]benzimidazole and 6,7-dihydro-2-[(4,5-dimethoxy-2-pyridyl)methylsulfinyl]-1H-[1,4]-dioxino[2,3-f]benzimidazole, and pharmaceutically acceptable salts of these compounds.

In another embodiment, the PPI is 2-((4-(3-methoxypropyl)-3-methylpyridin-2-yl)methylsulfinyl)-1H-benzimidazole as disclosed in U.S. Pat. Nos. 5,035,899 and 5,045,552.

In another embodiment, the PPI is (R)-2-(((3-methyl-4-(2,2,2-trifluoroethoxy)-2-pyridinyl)methyl)sulfinyl)-1H-benzimidazole as disclosed in U.S. Pat. Nos. 6,462,058, and 6,664,276.

The term “about” is used herein to mean the given number plus or minus 1 to 10%.

The term “individual” is used herein to refer to an animal and includes, for example, mammals such as humans, and veterinary animals such as sheep, elk, deer, horses, cattle, pigs, goats, dogs, cats, rats, mice, and birds.

In one embodiment, alkyl groups are “C₁-C₆ alkyl” groups which refers to a straight or branched saturated hydrocarbon chain having one to six carbon atoms and optionally substituted with one or more halo substituents or with one or more C₃-C₇ cycloalkyl groups. Examples include methyl, ethyl, n-propyl, isopropyl, t-butyl, n-hexyl, trifluoromethyl, 2-chloroethyl, methylenecyclopropyl, methylenecyclobutyl, methylenecyclobutyl and methylenecyclopentyl.

In one embodiment, “C₃-C₇ cycloalkyl” refers to a saturated 3 to 7 membered carbocyclic ring. Examples of such groups include cyclopropyl, cyclobutyl, cyclopentyl and cyclohexyl.

In one embodiment alkylene groups are “C₁-C₄ alkylene” groups which are disubstituted straight or branched saturated hydrocarbon chain having one to four carbon atoms.

“Halo” refers to fluoro, chloro, bromo or iodo.

In one embodiment, “aryl” refers to an aromatic ring system having from 5 to 14 ring carbon atoms and containing up to three rings. Examples of aryl groups are benzene and naphthalene.

In one embodiment “heteroaryl” refers to a ring system with aromatic character having from 5 to 14 ring atoms, at least one of which is a heteroatom selected from N, O and S, and containing up to three rings. Where a heteroaryl group contains more than one ring, not all rings must be fully aromatic in character. Rings which are not fully aromatic may be substituted with one or more oxo groups. Examples of heteroaryl groups include pyrrole, thiophene, thiazole, pyridine, pyrimidine, indole, benzofuran, benzimidazole, tetrahydroquinoline, indoline, quinoline, isoquinoline, quinoxaline, imidazo[1,2-a]pyridine, pyrazolo[1,5-a]pyridine, 2,3-dihydro-1-benzothiopyrane and 2,3-dihydro-1-benzothiopyran-1λ⁶-dione.

In one embodiment “heterocyclyl” refers to a saturated ring system having from 4 to 8 ring atoms, at least one of which is a heteroatom selected from N, O and S and which may be optionally substituted by one or more oxo groups. Examples of heterocyclyl groups include azetidinyl, piperidinyl; tetrahydrofuranyl, tetrahydropyranyl, dioxanyl, thiomorpholinyl, 1,1-dioxo-1,6-thiomorpholinyl, morpholinyl, pyrrolyl, piperizinyl, azepanyl, 1,4-diazepanyl, 1,4-oxazepanyl and azocanyl.

Appropriate pharmaceutically and veterinarily acceptable salts of the compounds of general formula (I) include basic addition salts such as sodium, potassium, calcium, aluminium, zinc, magnesium and other metal salts as well as choline, diethanolamine, ethanolamine, ethyl diamine, megulmine and other well-known basic addition salts as summarised in J. Med. Chem., 50, 6665-6672 (2007) and/or known to those skilled in the art.

Where appropriate, pharmaceutically or veterinarily acceptable salts may also include salts of organic acids, especially carboxylic acids, including but not limited to acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, pamoate, pectinate, 3-phenylpropionate, picrate, pivalate, proprionate, tartrate, lactobionate, pivolate, camphorate, undecanoate and succinate, organic sulfonic acids such as methanesulfonate, ethanesulfonate, 2-hydroxyethane sulfonate, camphorsulfonate, 2-naphthalenesulfonate, benzenesulfonate, p-chlorobenzenesulfonate and p-toluenesulfonate; and inorganic acids such as hydrochloride, hydrobromide, hydroiodide, sulfate, bisulfate, hemisulfate, thiocyanate, persulfate, phosphoric and sulfonic acids. Salts which are not pharmaceutically or veterinarily acceptable may still be valuable as intermediates.

If a chiral centre or another form of isomeric centre is present in a compound recited herein, all forms of such isomer or isomers, including enantiomers and diastereoisomers, are intended to be covered herein. Compounds containing a chiral centre may be used as a racemic mixture, an enantiomerically enriched mixture, or the racemic mixture may be separated using well-known techniques and an individual enantiomer may be used alone.

The term “preventing” is art-recognized and, when used in relation to esophagitis, includes administration of a composition which reduces the frequency of, or delays the onset of, symptoms of esophagitis in a subject relative to a subject which does not receive the composition. Thus, prevention of esophagitis includes, for example, reducing the difficulty of swallowing food (dysphagia), heartburn, chest pain, abdominal pain, nausea, vomiting, coughing, and failure to thrive in subjects.

The term “treating” includes reversing, reducing, or arresting the symptoms, clinical signs, and underlying pathology of esophagitis in a manner to improve or stabilize a subject.

In one embodiment, the CRTH2 antagonist and PPI are in the same pharmaceutical formulation. In another embodiment, the CRTH2 antagonist and the PPI are in separate pharmaceutical formulations.

The term “administered in combination with” refers to the co-administration of a CRTH2 antagonist with a PPI wherein the administration may be simultaneous, sequential, or separate.

Where the CRTH2 antagonist and the PPI are in separate formulations, administration of the CRTH2 antagonist may precede or follow the administration of the PPI by intervals ranging from minutes to hours. In one embodiment, the CRTH2 antagonist and the PPI may be administered within about 1 minute, about 5 minutes, about 10 minutes, about 30 minutes, about 60 minutes, about 2 hours, about 4 hours, about 6 hours, about 8 hours, about 10 hours, about 12 hours, about 18 hours, or about 24 hours of one another. In another embodiment, the CRTH2 antagonist and the PPI may be administered within about 1 minute, about 5 minutes, about 30 minutes, or about 60 minutes of one another.

In one embodiment, the CRTH2 antagonist and the PPI are administered according to the same dosing schedule. In another embodiment, the CRTH2 antagonist and the PPI are administered according to different dosing schedules. In one embodiment, the CRTH2 antagonist may be be administered twice a day while the PPI may be administered once a day. In another embodiment, the CRTH2 antagonist and the PPI are administered once a day.

The CRTH2 antagonist may be administered in dosages and according to dosing regimens known in the art. Dosages may range from about 0.01 mg to about 250 mg per day. In one embodiment, the CRTH2 antagonist may be administered in a dosage of 5, 10, 20, 30, 40, 50, 60, 70, 80, 90, 100, 110, 120, 130, 140, 150, 160, 170, 180, 190, 200, 210, 220, 230, 240, or 250 mg per day in single or divided dosages. In another embodiment, the dosage is 50, 70, or 100 mg administered once a day. In another embodiment, the dosage is 50, 70, or 100 mg administered twice a day. In another embodiment, a dosage level that is in the range of about 0.001 mg to about 10 mg per kg of body weight per day is employed. Variations in dosages may occur depending on the age, weight, and condition of the subject being treated, his or her individual response to the medicament, and the of pharmaceutical formulation and route of administration chosen, and the time period and interval during which such administration is carried out.

The PPI may be administered in dosages and according to dosing regimens known in the art. Dosages may range from about 0.01 mg to about 60 mg per day. In one embodiment, the PPI may be administered in a dosage of 5, 10, 15, 20, 30, 40, 50, 60, or 70 mg per day in single or divided dosages. In one embodiment, the PPI is omeprazole and the dosage is 10, 20, or 40 mg per day. In another embodiment, the PPI is lansoprazole and the dosage is 15 or 30 mg per day. In another embodiment, the PPI is rabeprazole and the dosage is 20 mg per day. In another embodiment, the PPI is pantoprazole and the dosage is 20 or 40 mg per day. In another embodiment, the PPI is esomeprazole and the dosage is 20 or 40 mg per day. In another embodiment, the PPI is dexlansoprazole and the dosage is 30 or 60 mg per day.

In one embodiment, the formulations as described herein may be synergistic in nature, meaning that the therapeutic effect of the combination of the CRTH2 antagonist and the PPI is greater than the sum of the individual effects.

In another embodiment, the formulations as described herein may be additive in nature, meaning that the therapeutic effect of the combination of the CRTH2 antagonist and the PPI is greater than the effect of each agent individually.

In one embodiment, the pharmaceutical formulation comprises (5-fluoro-2-methyl-3-quinolin-2-ylmethyl-indol-1-yl)-acetic acid, or a pharmaceutically acceptable salt thereof, and omeprazole, or a pharmaceutically acceptable salt thereof. In another embodiment, the pharmaceutical formulation comprises (5-fluoro-2-methyl-3-quinolin-2-ylmethyl-indol-1-yl)-acetic acid, or a pharmaceutically acceptable salt thereof, and lansoprazole, or a pharmaceutically acceptable salt thereof. In another embodiment, the pharmaceutical formulation comprises (5-fluoro-2-methyl-3-quinolin-2-ylmethyl-indol-1-yl)-acetic acid, or a pharmaceutically acceptable salt thereof, and rabeprazole, or a pharmaceutically acceptable salt thereof. In another embodiment, the pharmaceutical formulation comprises (5-fluoro-2-methyl-3-quinolin-2-ylmethyl-indol-1-yl)-acetic acid, or a pharmaceutically acceptable salt thereof, and pantoprazole, or a pharmaceutically acceptable salt thereof. In another embodiment, the pharmaceutical formulation comprises (5-fluoro-2-methyl-3-quinolin-2-ylmethyl-indol-1-yl)-acetic acid, or a pharmaceutically acceptable salt thereof, and esomeprazole, or a pharmaceutically acceptable salt thereof. In another embodiment, the pharmaceutical formulation comprises (5-fluoro-2-methyl-3-quinolin-2-ylmethyl-indol-1-yl)-acetic acid, or a pharmaceutically acceptable salt thereof, and dexlansoprazole, or a pharmaceutically acceptable salt thereof.

In one embodiment, the pharmaceutical formulation comprises [5-fluoro-3-(4-methanesulfonyl-benzyl)-2-methyl-indol-1-yl]-acetic acid, or a pharmaceutically acceptable salt thereof, and omeprazole, or a pharmaceutically acceptable salt thereof. In another embodiment, the pharmaceutical formulation comprises [5-fluoro-3-(4-methanesulfonyl-benzyl)-2-methyl-indol-1-yl]-acetic acid, or a pharmaceutically acceptable salt thereof, and lansoprazole, or a pharmaceutically acceptable salt thereof. In another embodiment, the pharmaceutical formulation comprises [5-fluoro-3-(4-methanesulfonyl-benzyl)-2-methyl-indol-1-yl]-acetic acid, or a pharmaceutically acceptable salt thereof, and rabeprazole, or a pharmaceutically acceptable salt thereof. In another embodiment, the pharmaceutical formulation comprises [5-fluoro-3-(4-methanesulfonyl-benzyl)-2-methyl-indol-1-yl]-acetic acid, or a pharmaceutically acceptable salt thereof, and pantoprazole, or a pharmaceutically acceptable salt thereof. In another embodiment, the pharmaceutical formulation comprises [5-fluoro-3-(4-methanesulfonyl-benzyl)-2-methyl-indol-1-yl]-acetic acid, or a pharmaceutically acceptable salt thereof, and esomeprazole, or a pharmaceutically acceptable salt thereof. In another embodiment, the pharmaceutical formulation comprises [5-fluoro-3-(4-methanesulfonyl-benzyl)-2-methyl-indol-1-yl]-acetic acid, or a pharmaceutically acceptable salt thereof, and dexlansoprazole, or a pharmaceutically acceptable salt thereof.

In one embodiment, the pharmaceutical formulation comprises (3-{[2-(benzenesulfonyl)pyridin-3-yl]methyl}-5-fluoro-2-methylindol-1-yl)-acetic acid, or a pharmaceutically acceptable salt thereof, and omeprazole, or a pharmaceutically acceptable salt thereof. In another embodiment, the pharmaceutical formulation comprises (3-{[2-(benzenesulfonyl)pyridin-3-yl]methyl}-5-fluoro-2-methylindol-1-yl)-acetic acid, or a pharmaceutically acceptable salt thereof, and lansoprazole, or a pharmaceutically acceptable salt thereof. In another embodiment, the pharmaceutical formulation comprises (3-{[2-(benzenesulfonyl)pyridin-3-yl]methyl}-5-fluoro-2-methylindol-1-yl)-acetic acid, or a pharmaceutically acceptable salt thereof, and rabeprazole, or a pharmaceutically acceptable salt thereof. In another embodiment, the pharmaceutical formulation comprises (3-{[2-(benzenesulfonyl)pyridin-3-yl]methyl}-5-fluoro-2-methylindol-1-yl)-acetic acid, or a pharmaceutically acceptable salt thereof, and pantoprazole, or a pharmaceutically acceptable salt thereof. In another embodiment, the pharmaceutical formulation comprises (3-{[2-(benzenesulfonyl)pyridin-3-yl]methyl}-5-fluoro-2-methylindol-1-yl)-acetic acid, or a pharmaceutically acceptable salt thereof, and esomeprazole, or a pharmaceutically acceptable salt thereof. In another embodiment, the pharmaceutical formulation comprises (3-{[2-(benzenesulfonyl)pyridin-3-yl]methyl}-5-fluoro-2-methylindol-1-yl)-acetic acid, or a pharmaceutically acceptable salt thereof, and dexlansoprazole, or a pharmaceutically acceptable salt thereof.

In one embodiment, the pharmaceutical formulation comprises [5-fluoro-3-({2-[(4-fluorobenzene)sulfonyl]pyridin-3-yl}methyl)-2-methylindol-1-yl]-acetic acid, or a pharmaceutically acceptable salt thereof, and omeprazole, or a pharmaceutically acceptable salt thereof. In another embodiment, the pharmaceutical formulation comprises [5-fluoro-3-({2-[(4-fluorobenzene)sulfonyl]pyridin-3-yl}methyl)-2-methylindol-1-yl]-acetic acid, or a pharmaceutically acceptable salt thereof, and lansoprazole, or a pharmaceutically acceptable salt thereof. In another embodiment, the pharmaceutical formulation comprises [5-fluoro-3-({2-[(4-fluorobenzene)sulfonyl]pyridin-3-yl}methyl)-2-methylindol-1-yl]-acetic acid, or a pharmaceutically acceptable salt thereof, and rabeprazole, or a pharmaceutically acceptable salt thereof. In another embodiment, the pharmaceutical formulation comprises [5-fluoro-3-({2-[(4-fluorobenzene)sulfonyl]pyridin-3-yl}methyl)-2-methylindol-1-yl]-acetic acid, or a pharmaceutically acceptable salt thereof, and pantoprazole, or a pharmaceutically acceptable salt thereof. In another embodiment, the pharmaceutical formulation comprises [5-fluoro-3-({2-[(4-fluorobenzene)sulfonyl]pyridin-3-yl}methyl)-2-methylindol-1-yl]-acetic acid, or a pharmaceutically acceptable salt thereof, and esomeprazole, or a pharmaceutically acceptable salt thereof. In another embodiment, the pharmaceutical formulation comprises [5-fluoro-3-({2-[(4-fluorobenzene)sulfonyl]pyridin-3-yl}methyl)-2-methylindol-1-yl]-acetic acid, or a pharmaceutically acceptable salt thereof, and dexlansoprazole, or a pharmaceutically acceptable salt thereof.

In one embodiment, the pharmaceutical formulation comprises 5-(acetylamino)-3-[(4-chlorophenyl)thio]-2-methyl-1H-indole-1-acetic acid, or a pharmaceutically acceptable salt thereof, and omeprazole, or a pharmaceutically acceptable salt thereof. In another embodiment, the pharmaceutical formulation comprises 5-(acetylamino)-3-[(4-chlorophenyl)thio]-2-methyl-1H-indole-1-acetic acid, or a pharmaceutically acceptable salt thereof, and lansoprazole, or a pharmaceutically acceptable salt thereof. In another embodiment, the pharmaceutical formulation comprises 5-(acetylamino)-3-[(4-chlorophenyl)thio]-2-methyl-1H-indole-1-acetic acid, or a pharmaceutically acceptable salt thereof, and rabeprazole, or a pharmaceutically acceptable salt thereof. In another embodiment, the pharmaceutical formulation comprises 5-(acetylamino)-3-[(4-chlorophenyl)thio]-2-methyl-1H-indole-1-acetic acid, or a pharmaceutically acceptable salt thereof, and pantoprazole, or a pharmaceutically acceptable salt thereof. In another embodiment, the pharmaceutical formulation comprises 5-(acetylamino)-3-[(4-chlorophenyl)thio]-2-methyl-1H-indole-1-acetic acid, or a pharmaceutically acceptable salt thereof, and esomeprazole, or a pharmaceutically acceptable salt thereof. In another embodiment, the pharmaceutical formulation comprises 5-(acetylamino)-3-[(4-chlorophenyl)thio]-2-methyl-1H-indole-1-acetic acid, or a pharmaceutically acceptable salt thereof, and dexlansoprazole, or a pharmaceutically acceptable salt thereof.

In one embodiment, the pharmaceutical formulation comprises a CRTH2 antagonist and a PPI without a corticosteroid. In another embodiment, the pharmaceutical formulation comprises a CRTH2 antagonist, a PPI, and a corticosteroid. In one embodiment, the corticosteroid is hydrocortisone, hydrocortisone acetate, cortisone acetate, tixocortol pivalate, prednisolone, methylprednisolone, or prednisone. In another embodiment, the corticosteroid is triamcinolone acetonide, triamcinolone alcohol, mometasone, amcinonide, budesonide, desonide, fluocinonide, fluocinolone acetonide, or halcinonide. In another embodiment, the corticosteroid is betamethasone, betamethasone sodium phosphate, dexamethasone, dexamethasone sodium phosphate, or fluocortolone. In another embodiment, the corticosteroid is hydrocortisone-17-valerate, aclometasone diproprionate, betamethasone valerate, betamethasone diproprionate, prednicarbate, clobetasone-17-butyrate, clobetasol-17-propionate, fluocortolone caproate, fluocortolone pivalate, or fluprednidene acetate. In another embodiment, the corticosteroid is hydrocortisone-17-butyrate, 17-aceponate, 17-buteprate, or prednicarbate.

In one embodiment, the pharmaceutical formulation comprises a CRTH2 antagonist and a PPI with an anti-IL-3 antibody. In one embodiment, the anti-IL-3 antibody is a monoclonal antibody. In a further embodiment, the anti-IL-3 antibody is a human or humanized monoclonal antibody. Anti-IL-3 antibodies are known and taught for example, by Lokker et al., J. Immunol. 146:893-898 (1991) and Finkelman et al., J. Immunol. 151:1235-1244 (1993).

In another embodiment, the pharmaceutical formulation comprises a CRTH2 antagonist and a PPI with montelukast.

In another embodiment, the present invention provides a maintenance therapy regimen for the treatment of eosinophilic esophagitis.

In one embodiment, the present invention provides a method for the maintenance therapy of eosinophilic esophagitis comprising:
(a) firstly administering to an individual in need of such treatment a therapeutically effect amount of a corticosteroid for a first predetermined period of time; and
(b) subsequently administering to the individual a therapeutically effective amount of at least one CRTH2 antagonist or a pharmaceutically acceptable salt thereof and at least one proton pump inhibitor or a pharmaceutically acceptable salt thereof for a second predetermined period of time.

The method of this invention comprises first administering to an individual in need thereof a therapeutically effective amount of a corticosteroid for a first predetermined period of time. In one embodiment, the corticosteroid is fluticasone. In another embodiment, the corticosteroid is budesonide. The corticosteroid may be administered as instructed according to the manufacturer of the particular corticosteroid used for this invention. In one embodiment, the corticosteroid is administered once a day. In another embodiment, the corticosteroid is administered twice a day. The duration for the first predetermined period can be determined by a person skilled in the art. In one embodiment of the invention, the first predetermined period of time is between 1 and 24 weeks, between 1 and 16 weeks, between 1 and 4 weeks, and between 1 and 3 weeks.

Doses of swallowed steroid to induce clinical remission are shown in Table 1. Remission is usually induced after treatment for 1-3 weeks. Oral viscous budesonide is the particular steroid. Straumann, A., et al., Clinical Gastroenterology and Hepatology 9:400-409 (2011) disclosed a double-blind trial whether reduction to a dose of 0.25 mg budesonide twice-a-day is sufficient to maintain remission compared to placebo. Budesonide is more effective than placebo but is only partially effective in suppressing tissue eosinophilia. Consequently, there is an unmet medical need for new treatments to maintain patients in clinical remission.

TABLE 1

| | Children (<10 years) | | Adolescents and adults | |
|---|---|---|---|---|
| Fluticasone | 88-440 | μg twice daily | 440-880 | μg twice daily |
| (from MDI) | | | | |
| Budesonide (oral | 0.5 | mg twice daily | 1 | mg twice daily |
| viscous formula- | | | | |
| tions) | | | | |

The method of this invention also comprises subsequently administering to an individual in need thereof a therapeutically effective amount of at least one CRTH2 antagonist and at least one PPI for a second predetermined period of time. In one embodiment, the at least one CRTH2 antagonist is selected from the group consisting of (5-fluoro-2-methyl-3-quinolin-2-ylmethyl-indol-1-yl)-acetic acid or a pharmaceutically acceptable salt thereof, [5-fluoro-3-(4-methanesulfonyl-benzyl)-2-methyl-indol-1-yl]-acetic acid or a pharmaceutically acceptable salt thereof, (3-{[2-(benzenesulfonyl)pyridin-3-yl]methyl}-5-fluoro-2-methylindol-1-yl)-acetic acid or a pharmaceutically acceptable salt thereof, [5-fluoro-3-({2-[(4-fluorobenzene)sulfonyl]pyridin-3-yl}methyl)-2-methylindol-1-yl]-acetic acid or a pharmaceutically acceptable salt thereof, and 5-(acetylamino)-3-[(4-chlorophenyl)thio]-2-methyl-1H-indole-1-acetic acid. In one embodiment, the PPI is selected from the group consisting of omeprazole, esomeprazole, lansoprazole, dexlansoprazole, pantoprazole, and rabeprazole, or a pharmaceutically acceptable salt thereof. In one embodiment, the administration of the at least one CRTH2 antagonist and at least one PPI may start within a period of between 0 and 30 days after terminating administration of the corticosteroid.

The at least one CRTH2 antagonist and the at least one PPI may be administered at the same time or at different times. In one embodiment, the administration of the at least one CRTH2 antagonist and the at least one PPI starts immediately after terminating administration of the corticosteroid. The CRTH2 antagonist may be administered as instructed according to the manufacturer of the particular CRTH2 antagonist used for this invention. In one embodiment, the CRTH2 antagonist is administered once a day. The PPI may be administered as instructed according to the manufacturer of the particular PPI used for this invention. In one embodiment, the PPI is administered once a day. In another embodiment, the PPI is administered twice a day.

The duration for the second predetermined period can be determined by a person skilled in the art. In one embodiment of the invention, the first predetermined period of time is between 1 and 24 weeks, between 1 and 16 weeks, between 1 and 4 weeks, and between 1 and 3 weeks.

The method of this invention also comprises subsequently administering to an individual in need thereof a therapeutically effective amount of at least one CRTH2 antagonist and at least one PPI and further administering a corticosteroid for a second predetermined period of time. In one embodiment, the dosage of the corticosteroid in the first predetermined period of time is higher than the dosage of the corticosteroid in the second predetermined period of time.

Pharmaceutical formulations comprising PPI's are known and described in the aforementioned patents. PPI's are known to be unstable to acid. Thus, oral formulations comprising PPI's may comprise an enteric coating which remains intact in the stomach, and dissolves in the intestinal tract. In one embodiment, a pharmaceutical formulation is in the form of an enterically coated tablet or granule comprising (1) a core comprising the PPI, (2) a first layer coated on the core, and (3) a second layer coated on the first layer which is an enteric coating. The core may comprise the PPI and a suitable excipient such as mannitol or lactose, and a binder such as hydroxypropylcellulose or polyvinylpyrrolidone. The first or intermediate layer may comprise a substantially water-insoluble film-forming material such as ethylcellulose and polyvinyl acetate and, optionally, an alkaline material such as an alkaline earth metal oxide or salt, e.g. magnesium oxide, silicic anhydride, calcium silicate, magnesium hydroxide, magnesium carbonate, aluminum hydroxide, calcium stearate and magnesium stearate. The enteric coating may comprise hydroxymethylcellulose phthalate, cellulose acetate phthalate, methacrylic acid/methyl methacrylate copolymer, and polyvinyl acetate phthalate. In one embodiment, both the PPI and the CRTH2 antagonist are present in the core. In another embodiment, the PPI and the CRTH2 antagonist are not in the core, but admixed with the enterically coated tablets or granules. In another embodiment, the admixed enterically coated tablets or granules are in a capsule.

The CRTH2 antagonists and PPIs may also be administered in a pharmaceutical formulation which may be a formulation suitable for oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art.

The formulation may be prepared by bringing into association the above defined active agents with a carrier. In general, the formulations are prepared by uniformly and intimately bringing into association the active agent with liquid carriers or finely divided solid carriers or both, and then if necessary shaping the product.

Formulations for oral administration in the present invention may be presented as: discrete units such as capsules, sachets, tablets, which may be chewable tablets, or lozenges, each containing a predetermined amount of the active agent; as a powder or granules; as fine particles for sprinkling over food; as a solution or a suspension of the active agent in an aqueous liquid or a non-aqueous liquid; or as an oil-in-water liquid emulsion or a water-in-oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

In one embodiment, the CRTH2 antagonist and the PPI may be in the same form (e.g., both may be administered as tablets) while in another embodiment, the CRTH2 antagonist and the PPI may be administered in different forms (e.g., one may be administered as a tablet and the other may be administered as an oral suspension).

In one embodiment, the invention provides a kit comprising a carrier means having in close confinement at least one CRTH2 antagonist and at least one PPI. The kit contains instructions to facilitate the administration of the CRTH2 antagonist and the PPI. In one embodiment, the carrier means is a blister pack. In another embodiment, the kit comprises a blister pack designed to contain one or more CRTH2 tablets, one or more PPI tablets, and instructions for administration. Exemplary blister packs are known in the art.

### EXAMPLES

Having now generally described this invention, the same will be understood by reference to the following examples which are provided herein for purposes of illustration only and are not intended to be limiting unless otherwise specified.

### Example 1

### 8 Week Study in Patients with Active Eosinophilic Esophagitis

Study Design

The study was a randomized, double-blind, placebo-controlled, single-center study of (5-fluoro-2-methyl-3-quinolin-2-ylmethyl-indo-1-yl)-acetic acid (OC000459) for 8 weeks in patients with active (≧20 eos/hpf and symptoms), corticosteroid-dependent, and/or -resistant eosinophilic esophagitis (EoE). The study compared patients taking 100 mg of OC000459 twice daily with patients taking a placebo twice daily. The study consisted of 26 patients with 14 patients taking OC000459 and 12 patients taking the placebo. Pre- and post-treatment disease-activity was assessed clinically, endoscopically, histologically, and via biomarkers. The primary endpoint was the reduction of the esophageal eosinophil load.

Study Population

The following selection criteria were used to identify subjects:

Inclusion Criteria:

1. Males and females ages 18-75 years.

2. Previously diagnosed and symptomatic isolated eosinophilic esophagitis.

3. Relevant eosinophil tissue inflammation as demonstrated by a mean eosinophil load ≧20 eos/hpf in 8 biopsies at the baseline visit.

4. Able to swallow placebo medication successfully under supervision in the clinic.

5. Free of all medications for EoE (including topical steroids) for at least 2 weeks prior to baseline and free of systemic steroids for at least 90 days before screening. A proton-pump inhibitor is allowed if required for treatment of secondary acid reflux.

Exclusion Criteria:

1. Other causes of esophagitis (GERD, peptic ulceration, and/or infection).

2. Other causes of esophagal or generalized eosinophilia (i.e., hypereosinophilic syndromes, parasitic infection, GERD).

3. The patient's EoE is dependent on the level of seasonal allergens and the patient's participation in the study will occur during the allergy season.

4. History of an abnormal gastric or duodenal eosinophilia (e.g., HES, Churg-Strauss vasculitis, EG, or a parasitic infection).

5. Receipt of a forbidden prescribed or over-the-counter medication within 4 weeks prior to the baseline visit and for the duration of the trial, including vitamins and herbal remedies.

Results

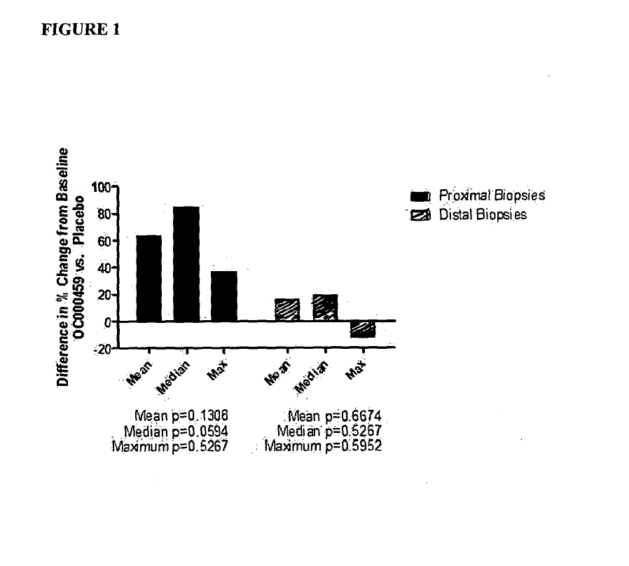

After an 8-week treatment of active EoE with OC000459, the total mean eosinophil number decreased from 114.7 to 74.2 eos/hpf, whereas under placebo, no reduction was observed (from 102.8 to 99.4 eos/hpf). However, the effect of drug was more pronounced in the proximal upper esophagus than in the distal esophagus. The difference in % change in eosinophil load compared to placebo is shown in FIG. 1.

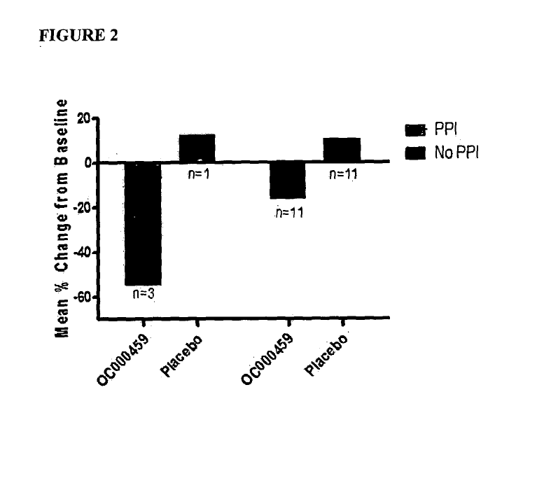

These data indicate that eosinophil infiltration in the upper esophagus may be mediated by CRTH2 but that eosinophil accumulation in the distal esophagus is CRTH2-resistant. A possible explanation for this is that acid reflux may be responsible for the eosinophilic inflammation in the distal esophagus which is consistent with reports that eosinophilia is reduced by PPIs in some patients with EoE (Molina-Infante et al., 2011). These data highlight two components of eosinophil accumulation in EoE, an allergic mechanism mediated by CRTH2 and acid reflux-dependent process which is reduced by PPI therapy. It is therefore proposed that the combination of CRTH2 antagonists with PPIs will be effective in the treatment of EoE by blocking both the allergic and acid reflux pathways. Three patients were treated with both OC000459 and esomeprazole, either 20 mg or 40 mg once a day. As shown in FIG. 2, these patients demonstrated a profound reduction in eosinophilic load compared to those patients taking OC000459 alone.

### CONCLUSIONS

OC00459 reduces eosinophilic load in the proximal but not distal esophagus in patients with EoE. When combined with a PPI to reduce acid reflux there is a considerable reduction in total eosinophilic load. Consequently, the combination of a CRTH2 antagonist with a PPI is an effective method to control inflammation of the esophagus in EoE which may be more convenient and safer than the current use of topical corticosteroids.

Having now fully described this invention, it will be understood by those of ordinary skill in the art that the same can be performed within a wide and equivalent range of conditions, formulations and other parameters without affecting the scope of the invention or any embodiment thereof. All patents, patent applications, and publications cited herein are fully incorporated by reference in their entirety.

1. A pharmaceutical composition comprising at least one CRTH2 antagonist or a pharmaceutically acceptable salt thereof and at least one proton pump inhibitor or a pharmaceutically acceptable salt thereof.

2. A pharmaceutical composition according to claim 1, wherein said CRTH2 antagonist is a compound of general formula (I): 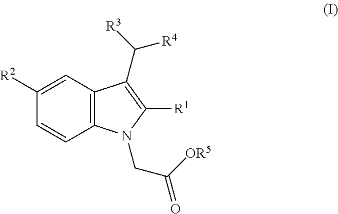
wherein
R¹ is C₁-C₆ alkyl;
R² is halogen;
R³ is aryl or heteroaryl optionally substituted with one or more substituents selected from halo, OH, CN, R⁶, COR⁶, CH₂R⁶, OR⁶, SR⁶, SO₂R⁶, or SO₂YR⁶;
R⁶ is C₁-C₆ alkyl, C₃-C₈ cycloalkyl, heterocyclyl, aryl, or heteroaryl, any of which may optionally be substituted with one or more substituents selected from halo, OH, CN, NO₂, C₁-C₆ alkyl, or O(C₁-C₆ alkyl); and
Y is NH or a straight or branched C₁-C₄ alkylene chain;
R⁴ is H or C₁-C₄ alkyl; and
R⁵ is hydrogen, C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
or a pharmaceutically acceptable salt, hydrate, solvate, or complex thereof.

3. A pharmaceutical composition, according to claim 2 wherein, in the compound of general formula (I), R⁵ is hydrogen.

4. A pharmaceutical composition, according to claim 3 wherein, in the compound of general formula (I), R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂.

5. A pharmaceutical composition according to any one of claims 2 to 4, wherein, in the compound of general formula (I), independently or in any combination:
R¹ is C₁-C₄ alkyl;
R² is fluoro;
R³ is optionally substituted and is quinoline, quinoxaline, isoquinoline, thiazole, phenyl, naphthalene, thiophene, pyrrole, or pyridine; and
R⁴ is H or methyl.

6. A pharmaceutical composition, method or use according to claim 2, wherein the compound of general formula (I) is:
{3-[1-(4-Chloro-phenyl)-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
[5-Fluoro-2-methyl-3-(1-naphthalen-2-yl-ethyl)-indol-1-yl]-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)-2-methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-1-yl]-acetic acid;
[5-Fluoro-2-methyl-3-(1,3-thiazol-2-ylmethyl)indol-1-yl]-acetic acid;
[3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-y)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-({1-[(4-methylbenzene)sulfonyl]pyrrol-2-yl}methyl)indol-1-yl]-acetic acid;
[3-({1-[(2,4-Difluorobenzene)sulfonyl]pyrrol-2-yl}methyl)-5-fluoro-2-methylindol-1-yl]-acetic acid;
(3-{[2-(Benzenesulfonyl)phenyl]methyl}-5-fluoro-2-methylindol-1-yl)-acetic acid;
[3-({2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{([2-(Benzenesulfonyl)pyridin-3-yl]methyl}-5-fluoro-2-methylindol-1-yl)-acetic acid;
[5-Fluoro-3-({2-[(4-fluorobenzene)sulfonyl]pyridin-3-yl}methyl)-2-methylindol-1-yl]-acetic acid;
[3-({2-[(4-Chlorobenzene)sulfonyl]pyridin-3-yl}methyl)-5-fluoro-2-methylindol-1-yl]-acetic acid;
2-(3-(4-(Benzylsulfonyl)benzyl)-5-fluoro-2-methyl-indol-1-yl)-acetic acid;
2-(3-(4-(4-Chlorobenzysulfonyl)benzyl)-5-fluoro-2-methyl-indol-yl)-acetic acid;
2-(3-(3-(Benzylsulfonyl)benzyl)-5-fluoro-2-methyl-indol-1-yl)-acetic acid;
2-(5-Fluoro-3-(3-(4-fluorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopenylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-1-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
{5-Fluoro-3-[(1-methanesulfonylnaphthalen-2-yl)methyl]-2-methylindol-1-yl}-acetic acid;
{5-Fluoro-3-[(6-methanesulfonylnaphthalen-2-yl)methyl]-2-methylindol-1-yl}-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]-acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-methylindol-1-yl}-acetic acid;
(5-Fluoro-2-methyl-3-{pyrazolo[1,5-a]pyridin-3-ylmethyl}indol-1-yl)-acetic acid;
(5-Fluoro-3-{imidazo[1,2-a]pyridin-2-ylmethyl}-2-methylindol-1-yl)-acetic acid;
(5-Fluoro-2-methyl-3-{[2-(methylsulfanyl)phenyl]methyl}indol-1-yl)-acetic acid;
(5-Fluoro-2-methyl-3-{[3-(methylsulfanyl)phenyl]methyl}indol-1-yl)-acetic acid;
(5-Fluoro-2-methyl-3-{[4-(ethylsulfanyl)phenyl]methyl}indol-1-yl)-acetic acid;
(3-{[4-(Ethylsulfanyl)phenyl]methyl}-5-fluoro-2-methylindol-1-yl)-acetic acid;
(5-Fluoro-2-methyl-3-{[4-(n-propylsulfanyl)phenyl]methyl}indol-1-yl)-acetic acid;
(5-Fluoro-2-methyl-3-{[4-(i-propylsulfanyl)phenyl]methyl}indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-{[4-(pentan-3-ylsulfanyl)phenyl]methyl}indol-1-yl)-acetic acid;
[3-({4-[(Cyclopropylmethyl)sulfanyl]phenyl}methyl)-5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-{[2-(Ethanesulfonyl)phenyl]methyl}-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-{[2-(Butene-2-sulfonyl)phenyl]methyl}-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-{[2-(pentane-1-sulfonyl)phenyl]methyl}indol-1-yl)-acetic acid;
(3-{[2-(Cyclopropylmethane)sulfonylphenyl]methyl}-5-fluoro-2-methylindol-1-yl)-acetic acid,
(5-Fluoro-2-methyl-3-{[2-(propylsulfamoyl)phenyl]methyl}indol-1-yl)-acetic acid;
(3-{[2-(Butylsulfamoyl)phenyl]methyl}-5-fluoro-2-methylindol-1-yl)-acetic acid;
(5-Fluoro-2-methyl-3-{[3-(propylsulfamoyl)phenyl]methyl}indol-1-yl)-acetic acid;
(3-{[3-(Butylsulfamoyl)phenyl]methyl}-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl}indol-1-yl)-acetic acid;
(5-Fluoro-2-methyl-3-{[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-{[4-(pentan-3-ylsulfonyl)phenyl]methyl}indol-1-yl)-acetic acid;
[3-({4-[(Cyclopropylmethyl)sulfonyl]phenyl}methyl)-5-fluoro-2-methylindol-1-yl]-acetic acid;
(5-Fluoro-2-methyl-3-{[4-(propylsulfamoyl)phenyl]methyl}indol-1-yl)-acetic acid;
(3-{[4-(Butylsulfamoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methylindol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzenesulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]-acetic acid;
{5-Fluoro-2-methyl-3-[(2-phenylphenyl)methyl]indol-1-yl}-acetic acid;
(3-{[1-(Benzenesulfonyl)indol-2-yl]methyl}-5-fluoro-2-methylindol-1-yl)-acetic acid;
(3-{[2-(4-Chlorophenyl)phenyl]methyl}-5-fluoro-2-methylindol-1-yl)-acetic acid;
(5-Fluoro-2-methyl-3-{[2-(4-methylphenyl)phenyl]methyl}indol-1-yl)-acetic acid;
{5-Fluoro-2-methyl-3-[(3-phenoxyphenyl)methyl)indol-1-yl}-acetic acid;
[5-Fluoro-3-({4-[(4-fluorophenyl)carbonyl]-1-methylpyrrol-2-yl}methyl)-2-methylindol-1-yl]-acetic acid;
{5-Fluoro-2-methyl-3-[(6-{[3-(trifluoromethyl)phenyl]methyl}pyridin-3-yl)methyl]indol-1-yl}-acetic acid;
{5-Fluoro-2-methyl-3-[(3-phenoxythiophen-2-yl)methyl]indol-1-yl}-acetic acid;
(3-{[2-(Benzenesulfonyl)-1,3-thiazol-5-yl]methyl}-5-fluoro-2-methylindol-1-yl)-acetic acid;
{3-[(1-Benzylpyrazol-4-yl)methyl]-5-fluoro-2-methylindol-1-yl}-acetic acid;
(3-{[5-(4-Chlorophenoxy)-1-methyl-3-(trifluoromethyl)pyrazol-4-yl]methyl}-5-fluoro-2-methylindol-1-yl)-acetic acid;
[3-({5-[(4-Chlorobenzene)sulfonyl]furan-2-yl}methyl)-5-fluoro-2-methylindol-1-yl]-acetic acid;
[3-({5-[(4-Chlorobenzene)sulfonyl]thiophen-2-yl}methyl)-5-fluoro-2-methylindol-1-yl]-acetic acid;
[3-({3-[(4-Chlorobenzene)sulfonyl]thiophen-2-yl}methyl)-5-fluoro-2-methylindol-1-yl]-acetic acid;
{3-[(2-Benzylphenyl)methyl]-5-fluoro-2-methylindol-1-yl}-acetic acid;
or a pharmaceutically acceptable salt thereof;
or the C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂ esters of any of the above; wherein
m is 1 or 2;
n is 1-4;
X is OR⁷ or N(R⁷)₂;
R⁷ is hydrogen or methyl; and
R⁸ is C₁-C₁₈ alkyl.

7. A pharmaceutical composition according to claim 6 wherein the CRTH2 antagonist is (5-Fluoro-2-methyl-3-quinolin-2-ylmethyl-indol-1-yl)-acetic acid; or a pharmaceutically acceptable salt thereof.

8. A pharmaceutical composition according to claim 6 wherein the CRTH2 antagonist is (3-{[2-(Benzenesulfonyl)pyridin-3-yl]methyl}-5-fluoro-2-methylindol-1-yl)-acetic acid; or
[5-Fluoro-3-({2-[(4-fluorobenzene)sulfonyl]pyridin-3-yl}methyl)-2-methylindol-1-yl]-acetic acid;
or a pharmaceutically acceptable salt thereof.

9. A pharmaceutical composition according to any one of claims 1 to 8, wherein the PPI is selected from the group consisting of omeprazole, esomeprazole, lansoprazole, dexlansoprazole, pantoprazole, and rabeprazole, or a pharmaceutically acceptable salt thereof.

10. A pharmaceutical composition according to claim 9, wherein:
(a) the CRTH2 antagonist is (5-fluoro-2-methyl-3-quinolin-2-ylmethyl-indol-1-yl)-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(b) the CRTH2 antagonist is [5-fluoro-3-(4-methanesulfonyl-benzyl)-2-methyl-indol-1-yl]-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(c) the CRTH2 antagonist is (3-{[2-(benzenesulfonyl)pyridin-3-yl]methyl}-5-fluoro-2-methylindol-1-yl)-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(d) the CRTH2 antagonist is [5-fluoro-3-({2-[(4-fluorobenzene)sulfonyl]pyridin-3-yl}methyl)-2-methylindol-1-yl]-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(e) the CRTH2 antagonist is 5-(acetylamino)-3-[(4-chlorophenyl)thio]-2-methyl-1H-indole-1-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof.

11. A pharmaceutical composition according to any one of claims 1 to 10, further comprising at least one corticosteroid; or at least one anti-IL-3 antibody.

12. A pharmaceutical composition according to claim 11, wherein the corticosteroid is selected from the group consisting of fluticasone, budesonide, hydrocortisone, dexamethasone, methylprednisolone, and prednisolone.

13. A pharmaceutical composition according to any one of claims 1 to 12, further comprising montelukast.

14. A product, comprising at least one CRTH2 antagonist or a pharmaceutically salt thereof and at least one proton pump inhibitor (PPI) or a pharmaceutical salt thereof for use in a method of preventing, treating, or ameliorating eosinophilic esophagitis (EoE).

15. A product for use according to claim 14 wherein the CRTH2 antagonist is as defined in any one of claims 2 to 8.

16. A product for use according to claim 14 or claim 15 wherein the PPI is selected from the group consisting of omeprazole, esomeprazole, lansoprazole, dexlansoprazole, pantoprazole, and rabeprazole, or a pharmaceutically acceptable salt thereof.

17. A product for use according to any one of claims 14 to 16 wherein the product comprises:
(a) the CRTH2 antagonist (5-fluoro-2-methyl-3-quinolin-2-ylmethyl-indol-1-yl)-acetic acid or a pharmaceutically acceptable salt thereof and a PPI selected from the group consisting of omeprazole, esomeprazole, lansoprazole, dexlansoprazole, pantoprazole, and rabeprazole, or a pharmaceutically acceptable salt thereof; or
(b) the CRTH2 antagonist [5-fluoro-3-(4-methanesulfonyl-benzyl)-2-methyl-indol-1-yl]-acetic acid or a pharmaceutically acceptable salt thereof and a PPI selected from the group consisting of omeprazole, esomeprazole, lansoprazole, dexlansoprazole, pantoprazole, and rabeprazole, or a pharmaceutically acceptable salt thereof; or
(c) the CRTH2 antagonist (3-{([2-(benzenesulfonyl)pyridin-3-yl]methyl}-5-fluoro-2-methylindol-1-yl)-acetic acid or a pharmaceutically acceptable salt thereof and a PPI selected from the group consisting of omeprazole, esomeprazole, lansoprazole, dexlansoprazole, pantoprazole, and rabeprazole, or a pharmaceutically acceptable salt thereof; or
(d) the CRTH2 antagonist [5-fluoro-3-({2-[(4-fluorobenzene)sulfonyl]pyridin-3-yl}methyl)-2-methylindol-1-yl]-acetic acid or a pharmaceutically acceptable salt thereof and a PPI selected from the group consisting of omeprazole, esomeprazole, lansoprazole, dexlansoprazole, pantoprazole, and rabeprazole, or a pharmaceutically acceptable salt thereof; or
(e) the CRTH2 antagonist 5-(acetylamino)-3-[(4-chlorophenyl)thio]-2-methyl-1H-indole-1-acetic acid or a pharmaceutically acceptable salt thereof and a PPI selected from the group consisting of omeprazole, esomeprazole, lansoprazole, dexlansoprazole, pantoprazole, and rabeprazole, or a pharmaceutically acceptable salt thereof.

18. A product for use according to any one of claims 14 to 18 wherein the CRTH2 antagonist and the PPI are for simultaneous, sequential or separate use.

19. A method of preventing, treating, or ameliorating eosinophilic esophagitis (EoE) in an individual, comprising administering to the individual a therapeutically effective amount of at least one CRTH2 antagonist or a pharmaceutically salt thereof and at least one proton pump inhibitor (PPI) or a pharmaceutical salt thereof.

20. A method according to claim 19 wherein the CRTH2 antagonist is as defined in any one of claims 2 to 8.

21. A method according to claim 19 or claim 20 wherein the PPI is selected from the group consisting of omeprazole, esomeprazole, lansoprazole, dexlansoprazole, pantoprazole, and rabeprazole, or a pharmaceutically acceptable salt thereof.

22. A method according to any one of claims 19 to 21 wherein:
(a) the CRTH2 antagonist is (5-fluoro-2-methyl-3-quinolin-2-ylmethyl-indol-1-yl)-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(b) the CRTH2 antagonist is [5-fluoro-3-(4-methanesulfonyl-benzyl)-2-methyl-indol-1-yl]-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(c) the CRTH2 antagonist is (3-{[2-(benzenesulfonyl)pyridin-3-yl]methyl}-5-fluoro-2-methylindol-1-yl)-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof, or
(d) the CRTH2 antagonist is [5-fluoro-3-({2-[(4-fluorobenzene)sulfonyl]pyridin-3-yl}methyl)-2-methylindol-1-yl]-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(e) the CRTH2 antagonist is 5-(acetylamino)-3-[(4-chlorophenyl)thio]-2-methyl-1H-indole-1-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof.

23. The use of a CRTH2 antagonist and a proton pump inhibitor (PPI) in the preparation of an agent for preventing, treating, or ameliorating eosinophilic esophagitis (EoE).

24. The use according to claim 23 wherein the CRTH2 antagonist is as defined in any one of claims 2 to 8.

25. The use according to claim 23 or claim 24 wherein the PPI is selected from the group consisting of omeprazole, esomeprazole, lansoprazole, dexlansoprazole, pantoprazole, and rabeprazole, or a pharmaceutically acceptable salt thereof.

26. The use according to any one of claims 23 to 25 wherein:
(a) the CRTH2 antagonist is (5-fluoro-2-methyl-3-quinolin-2-ylmethyl-indol-1-yl)-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(b) the CRTH2 antagonist is [5-fluoro-3-(4-methanesulfonyl-benzyl)-2-methyl-indol-1-yl]-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(c) the CRTH2 antagonist is (3-{[2-(benzenesulfonyl)pyridin-3-yl]methyl}-5-fluoro-2-methylindol-1-yl)-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(d) the CRTH2 antagonist is [5-fluoro-3-({2-[(4-fluorobenzene)sulfonyl]pyridin-3-yl}methyl)-2-methylindol-1-yl]-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof; or
(e) the CRTH2 antagonist is 5-(acetylamino)-3-[(4-chlorophenyl)thio]-2-methyl-1H-indole-1-acetic acid or a pharmaceutically acceptable salt thereof and the PPI is selected from the group consisting of omeprazole, esomeprazole, lansoprazole, dexlansoprazole, pantoprazole, and rabeprazole, or a pharmaceutically acceptable salt thereof.

27. A kit for the treatment of eosinophilic esophagitis comprising:
(a) at least one CRTH2 antagonist or a pharmaceutically acceptable salt thereof; and
(b) at least one proton pump inhibitor or a pharmaceutically acceptable salt thereof;
wherein the kit is packaged in one or more suitable containers.

28. A pharmaceutical composition according to any one of claims 1 to 13 or a product comprising at least one CRTH2 antagonist or a pharmaceutically acceptable salt thereof and at least one proton pump inhibitor or a pharmaceutically acceptable salt thereof for use in the maintenance therapy of eosinophilic esophagitis wherein the maintenance therapy comprises:
(a) firstly administering to an individual in need of such treatment a therapeutically effect amount of a corticosteroid for a first predetermined period of time; and
(b) subsequently administering to the individual a therapeutically effective amount of at least one CRTH2 antagonist or a pharmaceutically acceptable salt thereof and at least one proton pump inhibitor or a pharmaceutically acceptable salt thereof for a second predetermined period of time.

29. A product or a pharmaceutical composition for use according to claim 28, wherein the corticosteroid is budesonide.

30. A product or a pharmaceutical composition for use according to claim 28 or claim 29, wherein the corticosteroid is administered twice daily.

31. A product or a pharmaceutical composition for use according to any one of claims 28 to 30, wherein (b) further comprises administering a corticosteroid at a lower dosage than the dosage administered in (a).
